(19) United States
(12) Patent Application Publication
(10) Pub. No.: US 2024/0353394 A1
(43) Pub. Date: Oct. 24, 2024

(54) ASSAY METHODS FOR SCREENING INHIBITORS OF SICKLE CELL DISEASE, ß-THALASSEMIA, OR SICKLE CELL ß-THALASSEMIA, OR A PHENOTYPE THEREOF

(71) Applicant: Alexion Pharmaceuticals, Inc., Boston, MA (US)

(72) Inventors: Roxanne Cofiell, Glastonbury, CT (US); Sung-Kwon Kim, Cheshire (CT)

(73) Assignee: Alexion Pharmaceuticals, Inc., Boston, MA (US)

(21) Appl. No.: 18/682,330

(22) PCT Filed: Aug. 18, 2022
(86) PCT No.: PCT/US2022/040732

§ 371 Date: Feb. 8, 2024

Related U.S. Application Data
(60) Provisional application No. 63/235,290, filed on Aug. 20, 2021.
(60) Provisional application No. 63/349,277, filed on Jun. 6, 2022.

(51) Int. Cl. G01N 33/50

(52) U.S. Cl. CPC G01N 33/502; G01N 33/5047; G01N 33/5064; G01N 2333/4716; G01N 2800/22

(57) ABSTRACT

The present disclosure is directed to methods of identifying a test compounds for treating sickle cell disease (SCD), β-thalassemia (BT), or sickle cell BT. The methods involved contacting a test sample including cells with heme, serum, and a test compound, and measuring a biological phenomena including (1) deposition of a complement factor on the cells in the test sample; or (2) effect(s) of the complement factor deposition of (1) on target effector cells, where an attenuation in the biological phenomena in the test sample compared to the biological phenomena in a reference standard is indicative that the test compound is effective in treating sickle cell disease (SCD), -thalassemia (BT), or sickle cell BT.

### BACKGROUND OF THE INVENTION

Sickle cell disease (SCD) is a group of blood disorders characterized by an abnormality in the oxygen-carrying protein hemoglobin found in red blood cells leading to rigid, sickle-shaped red blood cells. Currently approved therapeutic drugs for treatment of SCD reduce one or more symptoms of SCD, e.g., pain and anemia, without addressing the underlying cause. Improved methods for screening inhibitors of SCD are needed in the field.

### SUMMARY OF THE INVENTION

The present disclosure is directed to methods of identifying test compounds for treating sickle cell disease (SCD), β-thalassemia (BT), or sickle cell BT.

The present disclosure relates to model system(s) and an assay(s) for analyzing the pathophysiology of sickle cell disease (SCD) at the cellular, tissue, and/or physiological levels. The disclosure is based, in part, on the finding that heme induces activation of complement pathway, particularly complement C3 and/or C5b9 deposition, on cells (e.g., sickle cell RBCs, endothall cells). Activation of the complement pathway elicits direct cellular damage and/or induces downstream processes such as changes in the cellular expression of surface proteins and/or recruitment of tissue factor (TF). The present model systems and assay methods are useful in examination of the effects of complement activation in situ on red blood cells (e.g., using ssRBC or induced ssRBC), endothelial cells (ECs), and blood cells (e.g., monocytes, neutrophils, and platelets), which can be used to benchmark the potential utility of complement pathway inhibitors such as C3 inhibitors, C5 inhibitors, FD inhibitors, FB inhibitors, properdin inhibitors, and other complement modulators, in the amelioration of the sickle cell phenotype. The assay methods of the disclosure provide a simple yet robust and high throughput method for analyzing modulation of various markers (e.g., iC3b and/or C5b9 deposition, including, EC marker expression and TF recruitment).

Under the model system developed herein, alternative pathway (AP) inhibitors, including factor P inhibitors (e.g., anti-properdin antibodies such as ALXN1820), oral factor B inhibitors such as iptacopan (LNP023), oral factor D inhibitors such as ALXN2050, complement C3 inhibitors (e.g., peptide inhibitors) and complement C5 inhibitors (e.g., anti-C5 antibodies such as N19/8), were identified as being therapeutically useful in the therapy of SCD as they were able to significantly inhibit the pathophysiology of SCD, such as complement targeting RBCs for destruction, endothelial activation, and tissue factor recruitment. Other modulators of the complement system, e.g., drugs that target complement C1q, complement C1, complement C1s, complement C2, MASP-2, MASP-3, Factor H, complement C5a/C5aR (receptor), complement C3a/C3aR (receptor), complement C6, and/or CD59, etc., can also be investigated for potential uses as drugs/medicaments for treating SCD.

In some embodiments, the present disclosure relates to a method of diagnosing SCD by measuring a change in biological phenomena in cells in response to heme and serum. The measured changes in biological phenomena may include, e.g., increased complement activation on cells in presence of heme and serum, e.g., increased C3 and/or C5b9 deposition in RBCs or endothelial cells (EC); or indirect effect of the increased complement deposition on target effector cells, e.g., increased activation of ECs or blood cells such as monocytes, neutrophils and/or platelets. Changes in activity and/or levels of complement proteins is indicative of a pathophysiology of SCD. Particularly, the present disclosure relates to a method of diagnosing SCD that includes imaging the complement deposition or measuring the activity thereof or downstream effects thereof in the presence of a complement modulator, e.g., inhibitor of alternative pathway of complement (AP). Composition deposition and/or measurement of the complement activity or downstream effects thereof may be carried out in a high throughput format using a fluorescence assay (FACS) or enzyme-linked immunosorbent assay (ELISA).

In some embodiments, the present disclosure relates to a method of screening for test compounds that are useful in the therapy of SCD by measuring ability of test compounds to reverse or attenuate changes in biological phenomena in cells that are elicited by heme and serum. The reversal or attenuation of the changes in biological phenomena may include, e.g., reversal or inhibition of increased complement activation on cells in presence of heme and serum, particularly, reversal or inhibition of increased C3 and/or C5b9 deposition in RBCs or endothelial cells (EC); or reversal or inhibition of increased activation of ECs or blood cells such as monocytes, neutrophils and/or platelets. As provided above, the ability of test compounds to reverse or attenuate the biological phenomena may be measured in a high-throughput format using FACS or ELISA assay.

In some embodiments, the present disclosure relates to a method of identifying a subpopulation of SCD patients responsive to therapy with complement inhibitors, e.g., inhibitors of AP. The method includes contacting a biological sample including cells (e.g., RBCs or endothelial cells) from a patient having or suspected of having SCD with heme and serum, optionally together with a complement inhibitor; measuring a change in biological phenomena in cells in the presence of complement inhibitor; and selecting samples which contain cells that undergo changes in the biological phenomena in response to the complement inhibitor; and identifying a subpopulation of SCD responsive to therapy with complement inhibitors based on the selected samples.

In some embodiments, the disclosure relates to methods of treatment of a patient having or suspected of having SCD. The method may involve making an assessment of the patient's SCD status, as provided above, e.g., measuring a change in biological phenomena in cells in response to heme and serum, such as, increased complement deposition in cells or perturbations in downstream effects of heme induced complement deposition on target effector cells; and treating the patient via administration of a compound that normalizes the altered biological phenomena. In some embodiments, the compound is a complement inhibitor, particularly an inhibitor of AP.

In some embodiments, the disclosure relates to use of a biological phenomenon elicited in cells (e.g., RBCs, ECs, or blood cells) in response to heme and serum, which phenomena is representative of SCD pathophysiology in the in vivo context, in the screening of test compounds that are useful in the treatment of SCD. Particularly, the use relates to identifying complement inhibitors such as AP inhibitors that can attenuate complement deposition in cells, including, activation of downstream effects of complement deposition on effector cells such as ECs and blood cells (e.g., monocytes, neutrophils, and platelets). In some embodiments, the disclosure relates to use of a biological phenomenon elicited in cells (e.g., RBCs, ECs, or blood cells) in response to heme and serum, in the diagnosis, classification, monitoring, and treatment of patients with SCD.

In some embodiments, the invention relates to a method of identifying a test compound for treating sickle cell disease (SCD), β-thalassemia (BT), or sickle cell BT is contemplated, the method including, contacting a test sample including cells with heme, serum, and the test compound; and measuring a biological phenomena including (1) deposition of a complement factor on the cells in the test sample; or (2) effect(s) of the complement factor deposition of (1) on target effector cells; wherein an attenuation in the biological phenomena in the test sample compared to the biological phenomena in a reference standard is indicative that the test compound is effective in treating sickle cell disease (SCD), β-thalassemia (BT), or sickle cell BT.

In some embodiments, the test sample includes red blood cells (RBCs), endothelial cells, or blood cells, or a combination thereof. In some embodiments, the blood cells are monocytes, neutrophils, and/or platelets. In some embodiments, deposition of the complement factor on RBCs and/or endothelial cells is measured. In some embodiments, the target effector cells are endothelial cells or blood cells. In some embodiments, the blood cells are monocytes, neutrophils and/or platelets.

In some embodiments, the reference standard includes an experimentally measured or predetermined level of a signal for the biological phenomena in a control sample that is devoid of the test compound. In some embodiments, the signal for the biological phenomena is a baseline C3 positivity level and/or a baseline C5b9 positivity level of about or greater than 20% in a population of endothelial cells. In some embodiments, the baseline C3 positivity level and/or a baseline C5b9 positivity level in the population of endothelial cells is greater than 30%. In some embodiments, the baseline C3 positivity level and/or a baseline C5b9 positivity level in the population of endothelial cells is greater than 50%. In some embodiments, the signal for the biological phenomena is a baseline Tissue Factor (TF) positivity level of about or greater than 10% in monocytes. In some embodiments, the baseline TF positivity level in monocytes is greater than 15%. In some embodiments, the baseline TF positivity level in monocytes is greater than 20%.

In some embodiments, the invention relates to a method of identifying a test compound for treating sickle cell disease (SCD), β-thalassemia (BT), or sickle cell BT, the method including, (a) contacting a first sample including cells with heme and serum; (b) contacting a second sample including the cells with the test compound, heme and serum, and (c) measuring a biological phenomena including (1) deposition of a complement factor on the cells in said first and the second samples; or (2) effect(s) of the complement deposition in the cells of said first and second samples on target effector cells; wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

In some embodiments, the first sample includes red blood cells (RBCs) or endothelial cells, or a combination thereof. In some embodiments, the blood cells are monocytes, neutrophils, and/or platelets. In some embodiments, the second sample includes RBCs or endothelial cells, or a combination thereof. In some embodiments, the blood cells are monocytes, neutrophils, and/or platelets. In some embodiments, the target effector cells are endothelial cells or blood cells, or a combination thereof. In some embodiments, the blood cells are monocytes, neutrophils and/or platelets.

In some embodiments, the biological phenomena is a baseline C3 positivity level and/or a baseline C5b9 positivity level of about or greater than 20% in a population of endothelial cells. In some embodiments, the baseline C3 positivity level and/or a baseline C5b9 positivity level in the population of endothelial cells is greater than 30%. In some embodiments, the baseline C3 positivity level and/or a baseline C5b9 positivity level in the population of endothelial cells is greater than 50%. In some embodiments, the biological phenomena is a baseline Tissue Factor (TF) positivity level of about or greater than 10% in monocytes. In some embodiments, the baseline TF positivity level in monocytes is greater than 15%. In some embodiments, the baseline TF positivity level in monocytes is greater than 20%.

In some embodiments, the invention relates to a method of identifying a test compound for treating sickle cell disease (SCD), β-thalassemia (BT), or sickle cell BT, the method including, (a) contacting a first sample including RBCs with heme and serum; (b) contacting a second sample including the RBCs with the test compound, heme, and serum, and (c) measuring a biological phenomena including (1) deposition of a complement factor on the RBCs in said first and the second samples; wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

In some embodiments, the invention relates to a method of identifying a test compound for treating sickle cell disease (SCD), β-thalassemia (BT), or sickle cell BT, the method including, (a) contacting a first sample including endothelial cells (ECs) with heme and serum for a period sufficient to induce complement deposition on ECs; (b) contacting a second sample including the ECs with the test compound, heme, and serum, and (c) measuring a biological phenomena including (1) deposition of a complement factor on the ECs in said first and the second samples; or (2) effect(s) of the complement deposition in the ECs of the first and second samples on target effector cells including ECs; wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

In some embodiments, the invention relates to a method of identifying a test compound for treating sickle cell disease (SCD), β-thalassemia (BT), or sickle cell BT, the method including, (a) contacting a first sample including blood cells such as monocytes, neutrophils, and/or platelets with heme and serum; (b) contacting a second sample including the blood cells with the test compound, heme, and serum, and (c) measuring a biological phenomena including effect(s) of deposition of a complement factor on the blood cells in said first and the second samples on target effector cells including blood cells; wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

In some embodiments, the measuring step includes flow cytometry.

In some embodiments, the complement factor includes complement factor C3, or a fragment thereof, or complement factor C5b9. In some embodiments, the complement factor is complement factor C3. In some embodiments, the complement factor is a protein fragment of complement factor C3. In some embodiments, the protein fragment of complement factor C3 is iC3b.

In some embodiments, the heme includes free heme provided at a concentration of 200 μM.

In some embodiments, the RBCs include sickle cell RBCs (ssRBC) or RBCs induced to form a ssRBC phenotype.

In some embodiments, the RBCs induced to form a ssRBC phenotype include cell-surface expression of phosphatidylserine (PS) or phosphatidylethanolamine (PE). In some embodiments, the RBCs include ssRBCs obtained from a sickle cell patient.

In some embodiments, the serum includes autologous serum. In some embodiments, the serum is from a sickle cell patient.

In some embodiments, the endothelial cells include dermal microvascular endothelial cells.

In some embodiments, the effect(s) of complement deposition on target effector cells is mediated via a complement receptor (CR) in effector cells. In some embodiments, the complement receptor is CR3 in monocytes. In some embodiments, the effect(s) of complement deposition on target effector cells results in upregulation of tissue factor (TF).

In some embodiments, the sample includes a blood sample. In some embodiments, the blood sample is a whole blood sample. In some embodiments, the blood sample includes an anti-coagulant. In some embodiments, the anti-coagulant is hirudin.

In some embodiments, the first and/or the second sample includes a blood sample. In some embodiments, the blood sample is a whole blood sample. In some embodiments, the blood sample includes an anti-coagulant. In some embodiments, the anti-coagulant is hirudin.

In some embodiments, the method further includes contacting a third sample including cells that have been contacted with heme and blood with an inhibitor of the complement alternative pathway (CAP).

In some embodiments, the cells are red blood cells (RBCs), endothelial cells (ECs), or blood cells. In some embodiments, the blood cells are monocytes, neutrophils and/or platelets.

In some embodiments, the CAP inhibitor includes a C3 inhibitor, a factor P inhibitor, a factor D (FD) inhibitor, or a C5 inhibitor. In some embodiments, the CAP inhibitor includes a peptide inhibitor of C3 or an oral factor D inhibitor. In some embodiments, the CAP inhibitor includes a properdin inhibitor. In some embodiments, the properdin inhibitor is an anti-properdin antibody. In some embodiments, the anti-properdin antibody is a bispecific antibody. In some embodiments, the bispecific anti-properdin antibody is a mini-body. In some embodiments, the CAP inhibitor includes a C5 inhibitor. In some embodiments, the C5 inhibitor is an anti-C5 antibody. In some embodiments, the anti-C5 antibody is eculizumab, ravulizumab, antibody 8110, or antibody N19-8 that binds specifically to human C5. In some embodiments, the anti-C5 antibody is a bispecific antibody. In some embodiments, the bispecific anti-C5 antibody is a mini-body.

In some embodiments, the method further includes contacting a control sample including cells that have been contacted with heme and blood with an inhibitor of P-selectin. In some embodiments, the cells are red blood cells (RBCs), endothelial cells (ECs), or blood cells, or a combination thereof. In some embodiments, the blood cells are monocytes, neutrophils and/or platelets. In some embodiments, the inhibitor of P-selectin is an antibody that binds to P-selectin. In some embodiments, the antibody is a monoclonal antibody. In some embodiments, the antibody is Crizanlizumab or has the amino acid sequence of Crizanlizumab.

In some embodiments, the contacting comprises administering heme, serum, and the test compound into a test animal. In some embodiments, the animal is a mouse, rat, guinea pig, rabbit, hamster, sheep, goat, monkey, or primate.

### BRIEF DESCRIPTION OF DRAWINGS

The patent or application file contains at least one drawing executed in color. Copies of this patent or patent application publication with color drawing(s) will be provided by the Office upon request and payment of the necessary fee.

For a more complete understanding of the principles disclosed herein, and the advantages thereof, reference is now made to the following descriptions taken in conjunction with the accompanying drawings.

FIG. 1 shows alternative pathway activation of sickle red blood cells in sickle cell disease pathophysiology.

FIG. 2A and FIG. 2B show benchmarking of alternative pathway inhibitors, including ALXN1820 (an anti-properdin antibody), ALXN2050 (a factor D inhibitor), and N19/8 (an anti-C5 antibody), against an antibody having the sequence of Crizanlizumab (an anti-P-selectin antibody) in heme-induced complement deposition on red blood cells. FIG. 2A shows heme-induced complement deposition of red blood cells for the protein C3. FIG. 2B shows heme-induced complement deposition of red blood cells for the protein C5b-9. FIG. 2A and FIG. 2B show that an antibody having the sequence of Crizanlizumab has little to no effect on C3 opsonization or C5b-9 deposition.

FIG. 3 shows that heme induces C3 deposition on SS-RBC's in a dose dependent fashion.

FIG. 4A and FIG. 4B show benchmarking of alternative pathway inhibitors, including ALXN1820, ALXN2050, and N19/8, against an antibody having the sequence of Crizanlizumab in heme-induced complement deposition on endothelial cells. FIG. 4A shows heme-induced complement deposition of endothelial cells for the protein C3. FIG. 4B shows heme-induced complement deposition of endothelial cells for the protein C5b-9. FIG. 4A and FIG. 4B show that ALXN1820 and ALXN2050 block heme-induced C3 and C5b-9.

FIG. 5A and FIG. 5B show AP Inhibitors blocking heme induced TF expression by monocytes in whole blood using flow cytometric analysis of monocytes from whole blood incubated with heme upregulated expression of TF, the initiator of extrinsic pathway of coagulation. FIG. 5A and FIG. 5B shows benchmarking of complement inhibitors, including ALXN1820, LNP023, a peptide C3 inhibitor, N19/8, ALXN2050, and a small molecule factor D (fD) inhibitor, against an antibody having the sequence of Crizanlizumab for the upregulation of tissue factor (TF). FIG. 5 shows that an antibody having the sequence of Crizanlizumab has no effect on TF upregulation by monocytes.

FIG. 6 shows IL-8 levels in a whole blood model of thromboinflammation when exposed to alterative pathway inhibitors, including ALXN1820, and Ec (anti C5), as compared to when the whole blood model of thromboinflammation is exposed to Crizanlizumab. FIG. 6 shows that an antibody having the sequence of Crizanlizumab has no effect on IL-8 production in the whole blood model of thromboinflammation.

FIG. 7A and FIG. 7B show microscopic imaging of P-selectin upregulation and complement deposition on endothelial cells treated with heme, respectively. FIG. 7A shows that P-selectin is upregulated by sickle cell disease relevant agonists. FIG. 7B shows complement deposition is induced by heme.

FIG. 8 shows flow cytometry-based data on heme-induced complement deposition on sickle RBCs and the effect of anti-properdin and anti-C5 antibody treatment. On the left are scatterplots showing iC3b deposition under various conditions, including normal, heme, heme+anti-properdin (ALXN1820), and heme+anti-C5. On the right is a bar graph quantifying the iC3b deposition. ****P<0.0001; **P<0.01.

FIG. 9 shows flow cytometry-based data on heme-induced complement deposition on sickle RBCs and the effect of anti-properdin (ALXN1820) and anti-C5 antibody treatment. On the left are scatterplots showing C5b9 levels under various conditions, including normal, heme, heme+anti-properdin, and heme+anti-C5. On the right is a bar graph quantifying the C5b9 levels. deposition. **P<0.01.

FIG. 10 shows bar charts showing flow cytometry-based analyses of heme-induced complement fragment deposition on endothelial cells exposed to heme and the effect of anti-properdin (ALXN1820) and anti-C5 monoclonal antibodies on complement deposition. Shown are changes in complement fragment levels, from left to right, normal, herne, heme+anti-properdin, and heme+anti-C5 pretreatment. The left-hand panel shows C3/C3b/iC3b deposition and the right-hand panel shows C5b9 deposition. ns=not significant. ****P<0.0001.

FIG. 11 shows an experimental outline for studying the effect of inhibition of complement activation in VOC in an in vivo mouse model of SCD. Townes SS mice are divided into five groups and prophylactically treated with PBS (vehicle), anti-properdin monoclonal antibodies (14E1), or anti-C5 (BB5.1) monoclonal antibodies four times from ten days before heme treatment. Animals were exposed to 50 μmol/Kg heme for three hours after which the animals were sacrificed. In one of the vehicle-treated group, animals were not exposed to heme and serve as a baseline. Upon euthanasia, blood samples and critical organs were harvested from animals to measure the level of complement deposition on RBCs, intravascular hemolysis and the severity of vaso-occlusions.

FIG. 12 shows bar charts showing effects of anti-properdin (14E1) and anti-C5 (BB5.1) antibodies against heme-induced intravascular hemolysis in SCD animals. Shown are changes in hemolysis marker levels, from left to right, under normal (control), heme, heme+14E1, and heme+BB5.1 pretreatment. The following hemolysis markers were measured: bilirubin (far left); lactate dehydrogenase (LDH) (center); and free hemoglobin (far right). ****P<0.0001; ***P<0.001; **P<0.01; *P<0.05; ns: not significant.

FIG. 13 shows bar charts showing effects of 14E1 and BB5.1 monoclonal antibodies against heme-induced intravascular hemolysis in SCD animals. Shown are changes in complement fragment levels, from left to right, normal, heme, heme+14E1, and heme+BB5.1 pretreatment. The left-hand panel shows C3/C3b/iC3b deposition and the right-hand panel shows C5b9 deposition. ***P<0.001; **P<0.01; *P<0.05; ns: not significant.

FIG. 14 shows data on heme-induced vaso-occlusion in the lung and the effect of 14E1 and BB5.1 monoclonal antibody treatment. On the left are representative photomicrographs of sickle cell (SS) RBCs in the lung of mice under the various conditions: normal (control), heme, heme+14E1, and heme+BB5.1 pretreatment. The right panel shows a bar graph quantifying fluorescence density of the images using standard software. ****P<0.0001; ***P<0.001.

FIG. 15 shows data on heme-induced vaso-occlusion in the liver and the effect of BB5.1 monoclonal antibody treatment. On the left are representative photomicrographs of sickle cell (SS) RBCs in the lung of mice under the various conditions: normal (control), heme, heme+14E1, and heme+BB5.1 pretreatment. The right panel shows a bar graph quantifying fluorescence density of the images using standard software. ****P<0.0001; ***P<0.001; *P<0.05.

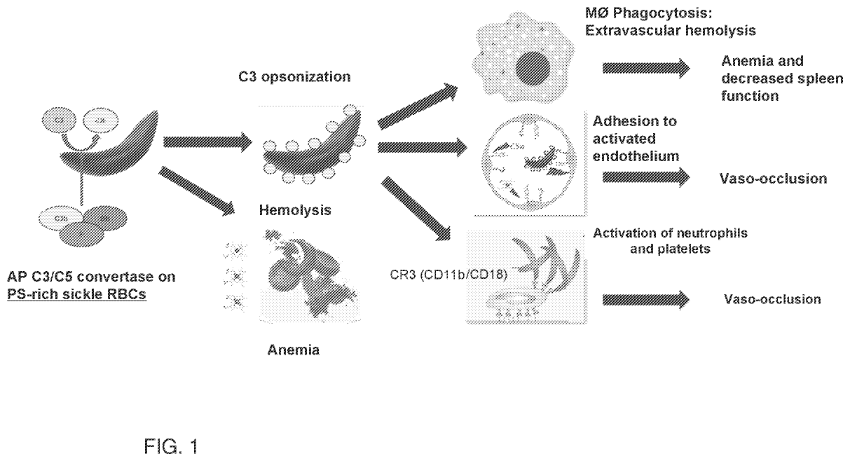

### DETAILED DESCRIPTION

The instant disclosure is based, in part, on the finding of the role of complement proteins, e.g., complement C5 and properdin, in the development and/or manifestation of Sickle cell disease (SCD), a life-threatening disease with poor quality of life for patients.

To assess proximal and terminal complement inhibition in the complement alternative pathway in vitro assays were developed to study complement blockade in heme induced deposition on sickle cell red blood cells (SS-RBCs) and the endothelial cell line, HMEC-1. Pre-treatment of cells with proximal AP inhibitors effectively inhibited opsonization and membrane attack complex (MAC) deposition on SS-RBCs and in endothelial cultures exposed to heme. Blockade of C5 effectively inhibited MAC deposition but not C3 deposition. Notably, crizanlizumab, an anti-P-selectin antibody had no effect on complement deposition.

A whole blood model of thromboinflammation was also developed in which heme and complement synergize to induce tissue factor (TF) expression on monocytes. The data presented in the Examples demonstrate that complement inhibition prevented TF expression while crizanlizumab did not.

### Definitions

Before describing the disclosure in detail, it is to be understood that this disclosure is not limited to particular compositions or biological systems, which can, of course, vary. It is also to be understood that the terminology used herein is for the purpose of describing particular embodiments only and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “and/or” includes any and all possible combinations of one or more of the associated listed items, as well as the lack of combinations when interpreted in the alternative (“or”).

It is understood that aspects and embodiments of the disclosure described herein include “comprising,” “consisting,” and “consisting essentially of” aspects and embodiments.

The term “about” means a range of plus or minus 10% of that value, e.g., “about 5” means 4.5 to 5.5, unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5.

The term “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance (e.g., +/−10%).

Where a range of values is provided in this disclosure, it is intended that each intervening value between the upper and lower limit of that range and any other stated or intervening value in that stated range is encompassed within the disclosure. For example, if a range of 1 mM to 8 mM is stated, it is intended that 2 mM, 3 mM, 4 mM, 5 mM, 6 mM, and 7 mM are also explicitly disclosed.

The term “subject” can be any animal, e.g., a mammal. A subject can be, for example, a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. Included are, e.g., transgenic animals or genetically altered (e.g., knock-out or knock-in) animals.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment, e.g., a particular therapeutic or prophylactic or diagnostic agent to treat a complement-mediated disease or disorder.

As used herein, the term “detecting,” refers to the process of determining a value or set of values associated with a sample by measurement of one or more parameters in a sample and may further include comparing a test sample against reference sample. In accordance with the present disclosure, the detection of SCD includes identification, assaying, measuring and/or quantifying one or more markers.

The term “sample” as used herein refers to a composition that is obtained or derived from a subject of interest that contains a cellular and/or other molecular entity that is to be characterized and/or identified, for example based on physical, biochemical, chemical and/or physiological characteristics. Preferably, the sample is a “biological sample,” which means a sample that is derived from a living entity, e.g., cells, tissues, organs, in vitro engineered organs, and the like. In some embodiments, the source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or 2D and 3D cultured cells or cell lines, cell supernatants, cell lysates, platelets, serum, plasma, and tissue culture medium, as well as tissue extracts such as homogenized tissue and cellular extracts. Samples further include biological samples that have been manipulated in any way after their procurement, such as by treatment with reagents (e.g., Ca2+ loading).

The term “label” as used herein refers, for example, to a compound that is detectable, either directly or indirectly. The term includes colorimetric (e.g., luminescent) labels, light scattering labels or radioactive labels. Fluorescent labels include, inter alia, the commercially available fluorescein phosphoramidites such as FLUOREPRIME™ (Pharmacia™), FLUOREDITE™ (Millipore™) and FAM™ (ABI™) (see, e.g., U.S. Pat. No. 6,287,778).

As used herein, the term “marker” refers to a characteristic that can be objectively measured as an indicator of normal biological processes, pathogenic processes or a pharmacological response to a therapeutic intervention, e.g., treatment with a drug/medicament for SCD. Representative types of markers include, for example, molecular changes in the structure (e.g., length of amino acid in a protein such as C3 or C5, e.g., due to proteolysis) or number of the marker, including, e.g., amount deposited in a cell, or a plurality of differences, such as both the levels as well as the activity of the markers of interest. The term “marker” includes both direct and indirect phenomena. For instance, wherein the analyte is C3, the marker could be C3 itself or a downstream effect of C3, e.g., terminal complement pathway protein such as C5 and effects thereof, e.g., C5 cleavage and C5b9 deposition.

The term “biological phenomena” as used herein refers to any processes that may be perturbed in a disease state, including, measurable changes therein in response to a test compound or an actual drug.

The term “screening”, as used herein, refers to an assay to assess the genotype or phenotype of a cell or cell product including, but not limited to, changes in the amount or structure or activity of a protein (e.g., levels of cleaved C3, particularly cleaved C3, and more particularly, convertase activity of C3). The assays include ELISA-based assays, BIACORE assays, activity assay (e.g., to measure C3 convertase activity) etc.

The term “positive”, as used herein, refers to identification of a parameter (e.g., the expression of an marker protein or activity thereof), which greater than by at least 5% (e.g., 10%, 20%, 30%, 50%, 75%, 100%, 200%, 300%, 500%, or more, e.g., 10-fold, 20-fold or 50-fold) of a control (e.g., expression of the same protein or activity thereof in a control cell, e.g., untreated cell).

The term “negative”, as used herein, refers to identification of a parameter (e.g., the expression of a protein or activity thereof), which less than 5% (e.g., 4%, 3%, 2%, 1%) of a control (e.g., expression of the same protein or activity thereof in a control cell, e.g., untreated cell). As used herein, the terms “treat” or “treating” refer to providing an intervention, e.g., providing any type of medical or surgical management of a subject. The treatment can be provided to reverse, alleviate, inhibit the progression of, prevent or reduce the likelihood of a disorder or condition, or to reverse, alleviate, inhibit, or prevent the progression of, prevent or reduce the likelihood of one or more symptoms or manifestations (e.g., pathophysiology) of a disorder or condition. “Prevent” refers to causing a disorder or condition, or symptom or manifestation of such not to occur for at least a period of time in at least some individuals. Treating can include administering a complement inhibitor (e.g., a complement C5 inhibitor) to the subject following the development of one or more symptoms or manifestations indicative of a complement-mediated condition, e.g., to reverse, alleviate, reduce the severity of, and/or inhibit or prevent the progression of the condition and/or to reverse, alleviate, reduce the severity of, and/or inhibit or one or more symptoms or manifestations of the condition. According to the methods described herein, a complement inhibitor (e.g., a C3 inhibitor, a factor P inhibitor, a factor D (FD) inhibitor, or a C5 inhibitor) can be administered to a subject who has developed a complement-mediated disease or is at increased risk of developing such a disorder relative to a member of the general population. Such an inhibitor (e.g., a C3 inhibitor, a factor P inhibitor, a factor D (FD) inhibitor, or a C5 inhibitor) can be administered prophylactically, i.e., before development of any symptom or manifestation of the condition. Typically, in this case, the subject will be at risk of developing the condition, for example, when exposed to a complement-activating condition, e.g., hypoxia.

The term “symptom” refers to an indication of disease, illness, injury, or that something is not right in the body. Symptoms are felt or noticed by the individual experiencing the symptom, but may not easily be noticed by others, e.g., non-health-care professionals. The term “sign” also refers an indication that something is not right in the body, which can be seen by a doctor, nurse, or other health care professional.

The terms “administration” or “administering” when used in conjunction with an agent, e.g., drug, mean to deliver the agent directly into or onto a cell or target tissue or to provide the agent to a patient whereby it impacts the tissue to which it is targeted.

The term “contact” refers to bringing an agent (e.g., an antibody, a nucleic acid molecule, a peptide, a small molecule, or an aptamer) and the target (e.g., C3, factor P, factor D, or C5) in sufficiently close proximity to each other for one to exert a biological effect on the other (e.g., inhibition of the target). In some embodiments, the term contact means binding of the agent to the target.

The terms “inhibitor” or “antagonist” as used herein refer to a substance, such as an antibody, nucleic acid, aptamer, and small molecule, that suppress the expression, activity, and/or level of another substance (e.g., complement C3, factor P, factor D, or C5). Functional or physiological antagonism occurs when two substances produce opposite effects on the same physiological function. Chemical antagonism or inactivation is a reaction between two substances to neutralize their effects, e.g., binding of an antibody to an antigen, which prevents the antigen from acting on its target. Dispositional antagonism is the alteration of the disposition of a substance (its absorption, biotransformation, distribution, or excretion) so that less of the agent reaches the target or its persistence there is reduced. The term “inhibit” or “reduce” or grammatical variations thereof refers to a decrease or diminishment in the specified level or activity of the target, e.g., little or essentially no detectible level or activity of the target (at most, an insignificant amount). Examples of inhibitors of this type are antibodies, interfering RNA molecules, such as siRNA, miRNA, and shRNA. The term “inhibitor of complement pathway” refers to inhibitors that suppress the activation of or response of the complement pathway.

The term “inhibitor of P-selectin” refers to inhibitors that suppresses the ability of P-selectin, a cell adhesion molecule (CAM), to interact with leukocytes. An inhibitor of P-selectin may be an antibody, such as a monoclonal antibody (e.g., crizanlizumab).

As used herein, the term “endogenous” describes a molecule (e.g., a metabolite, polypeptide, nucleic acid, or cofactor) that is found naturally in a particular organism (e.g., a human) or in a particular location within an organism (e.g., an organ, a tissue, or a cell, such as a human cell).

As used herein, the term “antibody” means an antibody, or a functional portion or fragment thereof, with a high binding affinity for an antigen, e.g., complement proteins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses natural, genetically engineered and/or otherwise modified antibodies of any class or subclass, including IgG and sub-classes thereof, IgM, IgE, IgA, and IgD.

The term “monoclonal antibody,” as used herein, refers to an antibody that displays a single binding specificity and affinity for a particular epitope. Accordingly, the term “human monoclonal antibody,” or “HuMab,” refers to an antibody that displays a single binding specificity, and that has variable and constant regions derived from human germline immunoglobulin sequences.

The term “single domain antibody”, also known as domain antibody, VHH, VNAR or sdAb, is a kind of antibody consisting of a single monomeric variable antibody domain and lacking the light chain and CH domain of the heavy chain in conventional Fab region. sdAbs can be generated from, e.g., VHH domains of camelid (e.g., dromedaries, camels, llamas, and alpacas) heavy-chain antibody and VNAR domains of cartilaginous fish (e.g., shark) heavy-chain antibody (known as immunoglobulin new antigen receptor (IgNAR)). Alternately, sdAbs may be generated by splitting dimeric variable domains from normal IgG of humans or mice into monomers by camelizing a few critical residues.

The term “bispecific” refers to a fusion protein of the disclosure that is capable of binding two antigens. The term “multivalent fusion protein” means a fusion protein including two or more antigen binding sites.

The term “small molecule” refers to an organic molecule having a molecular weight less than about 2500 amu, less than about 2000 amu, less than about 1500 amu, less than about 1000 amu, or less than about 750 amu. In some embodiments a small molecule contains one or more heteroatoms.

The term “aptamer” used herein refers to an oligonucleotide (generally, RNA molecule) linked to a specific target. “Aptamer” can refer to an oligonucleotide aptamer (for example, RNA aptamer). The term “aptamer” as used herein refers to DNA or RNA molecules that have been selected from random pools based on their ability to bind other molecules. Aptamers have been selected that bind nucleic acid, proteins, small organic compounds, and even entire organisms. A database of aptamers is maintained at world-wide-web at aptamer(dot)icmb(dot)utexas(dot)edu/.

As used herein, the term “complement C5” encompasses full-length, unprocessed complement C5, as well as any form of complement C5 resulting from processing in the cell, as well as any naturally occurring variants of complement C5 (e.g., splice variants or allelic variants). Human complement C5 has NCBI Gene ID NO 727. Exemplary wild-type human complement C5 nucleic acid sequences are provided in NCBI RefSeq Acc. No. NM_001317163.1 and NM_001735.2, and the respective exemplary wild-type complement C5 amino acid sequences are provided in NCBI RefSeq Acc. No. NP_001304092.1 and NP_001726.2.

As used herein, the term “complement C3” encompasses full-length, unprocessed complement C3, as well as any form of complement C3 resulting from processing in the cell, as well as any naturally occurring variants of complement C3 (e.g., splice variants or allelic variants). Human complement C5 has NCBI Gene ID NO 718.

As used herein, the term “human properdin” or “factor P” refers to a 469 amino acid soluble glycoprotein found in plasma that has seven thrombospondin type I repeats (TSR) with the N-terminal domain, TSR0, being a truncated domain. Human properdin, a 53 kDa protein, includes a signal peptide (amino acids 1-28), and six, non-identical TSR repeats about 60 amino acids each, as follows: amino acids 80-134 (TSR1), amino acids 139-191 (TSR2), amino acids 196-255 (TSR3), amino acids 260-313 (TSR4), amino acids 318-377 (TSR5), and amino acids 382-462 (TSR6). Properdin is formed by oligomerization of a rod-like monomer into cyclic dimers, trimers, and tetramers. The amino acid sequence of human properdin is found in the GenBank database under the following accession numbers: for human properdin, see, e.g., GenBank Accession Nos. AAA36489, NP-002612, AAH15756, AAP43692, S29126 and CAA40914. Properdin is a positive regulator of the alternative complement activation cascade. Known binding ligands for properdin include C3b, C3bB and C3bBb (Blatt, A. et al., Immunol. Rev., 274:172-90, 2016).

As used herein, the term “mouse properdin” refers to a 457 amino acid soluble glycoprotein found in plasma that has seven TSRs with the N-terminal domain, TSR0, being truncated. Mouse properdin, a 50 kDa protein, includes a signal peptide (amino acids 1-24), and six, non-identical TSRs of about 60 amino acids each, as follows: amino acids 73-130 (TSR1), amino acids 132-187 (TSR2), amino acids 189-251 (TSR3), amino acids 253-309 (TSR4), amino acids 311-372 (TSR5), and amino acids 374-457 (TSR6). Mouse properdin is formed by oligomerization of a rod-like monomer into cyclic dimers, trimers, and tetramers. The amino acid sequence of mouse properdin is found, for example, in the GenBank database (GenBank Accession Nos. P11680 and S05478).

The term “P-selectin” refers to type-1 transmembrane protein that in humans is encoded by the SELP gene and functions as a cell adhesion molecule (CAM) on the surfaces of activated endothelial cells, which line the inner surface of blood vessels, and activated platelets. P-selectin plays an essential role in the initial recruitment of leukocytes (white blood cells) to the site of injury during inflammation.

The term “red blood cells” or “RBCs” refer to cells circulating through the circulatory system responsible for carrying oxygen. Red blood cells may be induced to form a sickle cell phenotype, including cell-surface expression of phosphatidylserine (PS) or phosphatidylethanolamine (PE) via Ca2+ loading.

As used herein, the term “alternative complement pathway” refers to one of three pathways of complement activation (the others being the classical pathway and the lectin pathway). The alternative complement pathway is typically activated by bacteria, parasites, viruses or fungi, although IgA Abs and certain IgL chains have also been reported to activate this pathway.

As used herein, the term “alternative complement pathway dysregulation” refers to any aberration in the ability of the alternative complement pathway to provide host defense against pathogens and clear immune complexes and damaged cells and for immunoregulation. Alternative complement pathway dysregulation can occur both in fluid phase as well as at cell surface and can lead to excessive complement activation or insufficient regulation, both causing tissue injury.

The term “cell” refers to basic building blocks of tissue, such as cells from a human, monkey, mouse, rat, rabbit, hamster, goat, pig, dog, cat, ferret, cow, sheep, horse or the like. The cells may be diploid or haploid (i.e., sex cells). The cells may also be polyploid, aneuploid, or anucleate. The cell may be from a particular tissue or organ, such as blood, heart, lung, kidney, liver, bone marrow, pancreas, skin, bone, vein, artery, cornea, blood, small intestine, large intestine, brain, spinal cord, smooth muscle, skeletal muscle, ovary, testis, uterus, umbilical cord or the like. The cell may also be a platelet, myelocyte, erythrocyte, lymphocyte, adipocyte, fibroblast, epithelial cell, endothelial cell, smooth muscle cell, heart muscle, skeletal muscle cell, endocrine cell, glial cell, neuron, secretory cell, barrier function cell, contractile cell, absorptive cell, mucosal cell, limbus cell, stem cell (totipotent, pluripotent or multipotent), unfertilized or fertilized oocyte, sperm or the like. Included are normal cells and transformed cells.

The terms “sickle cell disease” or “SCD” have their general meaning in the art and refers to a hereditary blood disorder in which red blood cells assume an abnormal, rigid, sickle shape. Sickling of erythrocytes decreases the cells' flexibility and results in a risk of various life-threatening complications. The term includes sickle cell anemia, hemoglobin SC disease and sickle cell beta-thalassemia.

By “beta thalassemia” or “p thalassemia” as used herein is meant a hereditary blood disorder that is due to reduced or absent synthesis of the beta chains of hemoglobin. It is the result of one or more mutations in or near the β globin gene.

The term “intravenous” generally means “within a vein” and refers to accessing a subject's target cells or tissue via the vasculature system. In intravenous (IV) therapy, liquid substances are administered directly into a vein. Compared with other routes of administration, the intravenous route is probably the fastest way to deliver agents throughout a body. Some medications, blood transfusions, and parenteral (e.g., non-alimentary) nutrients are administered intravenously using standard delivery systems.

The terms “vaso-occlusion” or “VOC” have their general meaning in the art, e.g., relating to a common complication of SCD that leads to the occlusion of capillaries and the restriction of blood flow to an organ, resulting in ischemia, with vascular dysfunction, tissue necrosis, and/or organ damage. VOC are usually a constituent of vaso-occlusive crises, but they may also be more limited, clinically silent, and not cause hospitalization for vaso-occlusive crisis. As used herein, the term “vaso-occlusive crisis” refers to a painful complication of SCD that leads to hospitalization, in association with occlusion of capillaries and restriction of blood flow to an organ resulting in ischemia, severe pain, necrosis, and organ damage.

The term “acute chest syndrome” is a condition typically characterized by fever, chest pain, and appearance of a new infiltrate on chest radiograph. The term “chronic lung disease” in the context of SCD typically manifests as radiographic interstitial abnormalities, impaired pulmonary function, and, in its most severe form, by the evidence of pulmonary hypertension.

The term “hemolytic disease” refers to any disorder or disease in which cellular lysis, cellular damage and inflammation play a role in the pathology of the disease. Hemolytic disease is also an inflammatory disorder or disease wherein alternate pathway (AP) activation causes cellular lysis, cellular damage, and inflammation. Hemolytic diseases include diseases characterized by pathologic lysis of erythrocytes and/or platelets. Anucleated cells such as erythrocytes and platelets are subject to full lysis. Lysis of erythrocytes releases many markers, e.g., heme, hemoglobin, LDH, bilirubin, some of which may have pathological outcome for blood and organs. Nucleated cells such as neutrophils, monocytes, T lymphocytes can be attacked by the MAC but do not undergo full lysis. The term “intravascular hemolysis” refers to the lysis of anucleated and nucleated cells that is caused by AP activation and the associated production and deposition of C5b-9 on cell surfaces. The term “extravascular hemolysis” refers to lysis of cells due to C3b deposition and removal via complement receptors. C3b is produced via the activation of the classical and the alternative pathway. This disclosure relates to C3b produced via the alternative complement pathway.

The term “hemolytic anemia” as used herein refers to any condition in which the number of erythrocytes (RBC) per mm or the amount of hemoglobin in 100 mL of blood is less than normal, e.g., resulting from the destruction of erythrocytes. The term “thrombocytopenia” as used herein refers to a condition in which the number of platelets circulating in the blood is below the normal range of platelets.

The term “complement deposition” refers to an activity or event that leads to a complement component, e.g., C5b9 and/or C3, to deposit on a target cell (e.g., RBC or endothelial cell) by such a manner as to trigger a series of cascades (complement activation pathways) containing complement-related protein groups in blood. In addition, protein fragments generated by the activation of a complement can induce the migration, phagocytosis and activation of immune cells. Related downstream events include, e.g., (a) hemolysis of target cells, leading to heme release and/or anemia in blood cells; or (b) C3 opsonization, which may lead to phagocytosis and extra-vascular hemolysis (EVH); adhesion of opsonized cells to activated endothelium; and/or activation of neutrophils and platelets.

The term “trigger” in the context of SCD include any events or phenomena that initiate, propagate, or exacerbate disease symptom or pathology such as vaso-occlusive crises. Representative examples include, e.g., acidosis, hypoxia and dehydration, all of which potentiate intracellular polymerization of SS hemoglobin (J. H. Jandl, Blood: Textbook of Hematology, 2^{nd} Ed., Little, Brown and Company, Boston, 1996, pages 544-545).

As used herein, the term “marker” refers to a characteristic that can be objectively measured as an indicator of normal biological processes, pathogenic processes, or a pharmacological response to a therapeutic intervention, e.g., treatment with a complement inhibitor. Representative types of markers include, for example, molecular changes in the structure (e.g., sequence or length) or number of the marker, including, e.g., changes in level, concentration, activity, or properties of the marker.

The term “control” or “reference standard,” as used herein, refers to a reference for a test sample, such as control healthy subjects or untreated subjects, and the like. A “reference sample,” as used herein, refers to a sample of tissue or cells that may or may not have a disease that are used for comparisons. Thus a “reference” sample thereby provides a basis to which another sample, for example, blood from SCD patient, can be compared. In contrast, a “test sample” refers to a sample compared to a reference sample. The reference sample need not be disease free, such as when reference and test samples are obtained from the same patient separated by time.

The term “level” can refer to binary (e.g., absent/present), qualitative (e.g., absent/low/medium/high), or quantitative information (e.g., a value proportional to number, frequency, or concentration) indicating the presence of a particular molecular species. By a “decreased level” or an “increased level” of a protein or nucleic acid (e.g., mRNA) is meant a decrease or increase in protein or nucleic acid (e.g., mRNA) level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or nucleic acid (e.g., mRNA) in a sample.

The term “compounds” used in screening include any small molecule or large molecule compounds. The term “small molecule” includes compounds that are typically smaller than 5 KDa, e.g., organic compounds, peptides, aptamers, etc. The term “large molecule” includes compounds that are typically larger than 5 KDa, e.g., proteins and antibodies. Compounds may include agents known to have desired biological effects, e.g., reduce vaso-occlusion in SCD.

The term “pharmaceutical composition” refers to a preparation that is in such form as to permit the biological activity of an active ingredient contained therein to be effective, and that contains no additional components that are unacceptably toxic to a subject to which the formulation would be administered.

The term “attenuation” refers to the reduction of the force, effect, or value, as compared to a reference (e.g., a decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, as compared to a reference).

### Complement System in Pathology

The complement system acts in conjunction with other immunological systems of the body to defend against intrusion of cellular and viral pathogens. While a properly functioning complement system provides a robust defense against infecting microbes, inappropriate regulation or activation of the complement pathways has been implicated in the pathogenesis of a variety of disorders.

For example, the first report that complement activation may be involved in SCD was first published in 1967 (Francis and Womack. Am. J. Med. Technol. 1967; 33(2):77-86). Since then, studies have reported increased levels of complement-derived fragments in the blood of SCD patients, demonstrating that complement is activated in SCD and suggesting that complement may play an important role in the pathophysiology of the disease.

SCD pathology is known to arise from a missense mutation within the β-globin gene, leading to the substitution of valine for glutamic acid on the outer surface of the globin molecule. This amino acid substitution renders the sickle cell hemoglobin (HbS) less soluble and prone to polymerization upon deoxygenation. Erythrocytes (e.g., red blood cells; RBC) carrying polymerized HbS are thus less deformable and may obstruct microvessels. This vascular occlusion, producing tissue ischemic and infarction, represents a major cause of morbidity and mortality among SCD patients. Clinical manifestations of SCD extend far beyond the homozygous globin mutation. Seminal findings were the discovery that sickle (SS) RBCs, unlike normal RBCs, can adhere to stimulated endothelium in vitro and that SS-RBCs' adhesion correlates with the clinical severity of SCD. Subsequent studies have recognized the importance of plasma factors, such as complement proteins, in SS-RBC adhesion to the endothelium. In model systems of SCD, it has been shown that one of the complement proteins, C5a, is activated following the induction of hypoxia/re-oxygenation (e.g., see Vercellotti et al., Am. J. Hematol, 94:3 (2019), 327-338), further suggesting that complement proteins may be directly involved in the pathogenesis of this disorder. Importantly, however, the direct causal role of the complement system in the pathogenesis of SCD or a model thereof has yet to be demonstrated.

Mutations in the β-globin gene also cause other pathologies, including, for example, beta thalassemia (BT). Whereas BT major is caused by both alleles of the beta-globin gene containing a mutation that leads to complete absence of beta globin production, BT intermedia is due to reduced production of beta globin chains and/or production of mutant beta globin chains. BT is a disease that causes chronic anemia (e.g., a shortage of RBCs), which may suggest that complement proteins play an additional role in the pathogenesis of the genetically related disorder BT.

### Complement System in Pathology

The complement system acts in conjunction with other immunological systems of the body to defend against intrusion of cellular and viral pathogens. While a properly functioning complement system provides a robust defense against infecting microbes, inappropriate regulation or activation of the complement pathways has been implicated in the pathogenesis of a variety of disorders including, e.g., rheumatoid arthritis (RA); lupus nephritis; asthma; ischemia-reperfusion injury; atypical hemolytic uremic syndrome (aHUS); dense deposit disease (DDD); paroxysmal nocturnal hemoglobinuria (PNH); macular degeneration (e.g., age-related macular degeneration (AMD)); hemolysis, elevated liver enzymes and low platelets (HELLP) syndrome; Guillain-Barre Syndrome (GBS); protein-losing enteropathy (e.g., CHAPLE syndrome); myasthenia gravis (MG); neuromyelitis optica (NMO); post-hematopoietic stem cell transplant thrombotic microangiopathy (post-HSCT-TMA); post-bone marrow transplant TMA (post-BMT TMA); Degos disease; Gaucher's disease; glomerulonephritis; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis (Holers, V., Immunol. Rev., 223:300-16, 2008).

For example, the first report that complement activation may be involved in Sickle cell disease (SCD) was first published in 1967 e.g., see Francis and Womack. Am. J. Med. Technol. 1967; 33(2):77-86. Since then, studies have reported increased levels of complement-derived fragments in the blood of SCD patients, demonstrating that complement is activated in SCD and suggesting that complement may play an important role in the pathophysiology of the disease.

SCD pathology is known to arise from a missense mutation within the β-globin gene, leading to the substitution of valine for glutamic acid on the outer surface of the globin molecule. This amino acid substitution renders the sickle cell hemoglobin (HbS) less soluble and prone to polymerization upon deoxygenation. Erythrocytes (e.g., red blood cells; RBC) carrying polymerized HbS are thus less deformable and may obstruct microvessels. This vascular occlusion, producing tissue ischemic and infarction, represents a major cause of morbidity and mortality among SCD patients. Clinical manifestations of SCD extend far beyond the homozygous globin mutation. Seminal findings were the discovery that sickle (SS) RBCs, unlike normal RBCs, could adhere to stimulated endothelium in vitro and that SS-RBCs' adhesion correlated with the clinical severity of SCD. Subsequent studies have recognized the importance of plasma factors, such as complement proteins, in SS-RBC adhesion to the endothelium. In model systems of SCD, it has been shown that one of the complement proteins, C5a, is activated following the induction of hypoxia/re-oxygenation (e.g., see Vercellotti et al., Am. J. Hematol, 94:3 (2019), 327-338), further suggesting that complement proteins may be directly involved in the pathogenesis of this disorder. Importantly, however, the direct causal role of the complement system in the pathogenesis of SCD or a model thereof has yet to be demonstrated.

Mutations in the β-globin gene are also known to cause other pathologies, including beta thalassemia (BT). Whereas BT major is caused by both alleles of the beta-globin gene containing a mutation that leads to complete absence of beta globin production, BT intermedia is due to reduced production of beta globin chains and/or production of mutant beta globin chains. BT is a disease that causes chronic anemia (e.g., a shortage of RBCs), which may suggest that complement proteins could also play an additional role in the pathogenesis of the genetically related disorder BT.

### Complement Proteins

There are at least 25 complement proteins, which are a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory and lytic functions.

The complement cascade can progress via the classical pathway (CP), the lectin pathway, or the alternative pathway (AP). The CP is typically initiated by antibody recognition of, and binding to, an antigenic site on a target cell. The lectin pathway is typically initiated with binding of mannose-binding lectin (MBL) to high mannose substrates. The AP can be antibody independent and initiated by certain molecules on pathogen surfaces. These pathways converge at the C3 convertase—where complement component C3 is cleaved by an active protease to yield C3a and C3b.

Spontaneous hydrolysis of complement component C3, which is abundant in the plasma fraction of blood, can also lead to AP C3 convertase initiation. This process, known as “tickover,” occurs through the spontaneous cleavage of a thioester bond in C3 to form C3i or C3(H₂O). Tickover is facilitated by the presence of surfaces that support the binding of activated C3 and/or have neutral or positive charge characteristics (e.g., bacterial cell surfaces). Formation of C3(H₂O) allows for the binding of plasma protein Factor B, which in turn allows Factor D to cleave Factor B into Ba and Bb. The Bb fragment remains bound to C3 to form a complex containing C3(H₂O)Bb- the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). Factor B bound to the surface-bound C3b is cleaved by Factor D to form the surface-bound AP C3 convertase complex containing C3b,Bb.

The AP C5 convertase ((C3b)₂,Bb) is formed upon addition of a second C3b monomer to the AP C3 convertase. The role of the second C3b molecule is to bind C5 and present it for cleavage by Bb. The AP C3 and C5 convertases are stabilized by the addition of the trimeric protein properdin. Properdin binding, however, is not required to form a functioning alternative pathway C3 or C5 convertase.

The CP C3 convertase is formed upon interaction of complement component C1, which is a complex of C1q, C1r and C1s, with an antibody that is bound to a target antigen (e.g., a microbial antigen). The binding of the C1q portion of C1 to the antibody-antigen complex causes a conformational change in C1 that activates C1r. Active C1r then cleaves the C1-associated C1s to generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase (C4b, C2a, C3b) is formed upon addition of a C3b monomer to the CP C3 convertase.

In addition to its role in C3 and C5 convertases, C3b also functions as an opsonin through its interaction with complement receptors present on the surfaces of antigen-presenting cells such as macrophages and dendritic cells. The opsonic function of C3b is generally considered one of the most important anti-infective functions of the complement system. Patients with genetic lesions that block C3b function are prone to infection by a broad variety of pathogenic organisms, while patients with lesions later in the complement cascade sequence, e.g., patients with lesions that block C5 functions, are found to be more prone only to Neisseria infection, and then only somewhat more prone.

The AP and CP C5 convertases cleave C5 into C5a and C5b. Cleavage of C5 releases C5b, which allows for the formation of the lytic terminal complement complex, C5b-9. C5b combines with C6, C7 and C8 to form the C5b-8 complex at the surface of the target cell. Upon binding of several C9 molecules, the membrane attack complex (MAC, C5b-9, terminal complement complex (“TCC”)) is formed. When sufficient numbers of MACs insert into target cell membranes, the openings they create (MAC pores) mediate rapid osmotic lysis of the target cells.

Cleavage of C5 also releases C5a, which, has been shown to be potent anaphylatoxin and chemotactic factor.

### Complement Pathway Inhibitors

Compounds that bind to and inhibit a component of the complement pathway may be useful for treating SCD, BT, or sickle cell BT. The disclosed assays may be used to test compounds that bind to and inhibit a complement protein (e.g., C3, factor P (properdin), factor D, or C5) and are useful for treating SCD, BT, or sickle cell BT.

A test compound for an alternative complement pathway inhibitor can be selected from a number of different modalities. A test compound can be an antibody, a nucleic acid molecule (e.g., DNA molecule or RNA molecule, e.g., mRNA or inhibitory RNA molecule (e.g., short interfering RNA (siRNA), micro RNA (miRNA), or short hairpin RNA (shRNA)), or a hybrid DNA-RNA molecule), a peptide, a small molecule (e.g., a properdin small molecule inhibitor), an inhibitor of a signaling cascade, an activator of a signaling cascade, or an epigenetic modifier), or an aptamer. Any of these modalities can be a complement inhibitor directed to target (e.g., to inhibit) function of a complement protein; complement expression; complement binding; or complement signaling. The nucleic acid molecule or small molecule may include a modification. For example, the modification can be a chemical modification, e.g., conjugation to a marker, e.g., fluorescent marker or a radioactive marker. The modification can also include conjugation to an antibody to target the agent to a particular cell or tissue. Additionally, the modification can be a chemical modification, packaging modification (e.g., packaging within a nanoparticle or microparticle), or targeting modification.

Complement pathway inhibitors used in the assays described herein include ALXN1820, which is a bi-specific fusion molecule that binds properdin and human serum albumin; ALXN2050, also known as vermincopan, which is a small molecule factor D inhibitor; N19/8 which is an anti-C5 antibody (see, e.g., Wurzner et al. Inhibition of terminal complement complex formation and cell lysis by monoclonal antibodies, Complement Inflammation, 8:328-340 1991); and Iptacopan, which is also known as LNP023, is a small-molecule factor B inhibitor. Iptacopan has the CAS #1644670-37-0 and the FDA Drug No. 8E05T07Z6W.

Other complement inhibitors include Compounds 3 and 4, which are oral factor D (FD) inhibitors and have the structures:

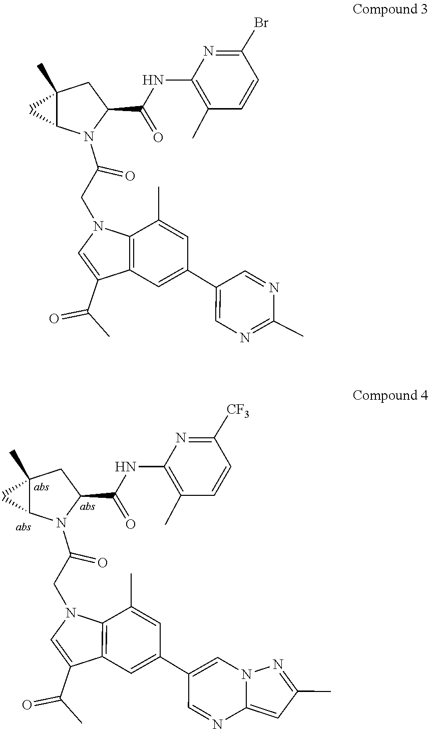 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12 ; CC(=O)c1cn(CC(=O)N2[C@H](C(=O)Nc3nc(C(F)(F)F)ccc3C)C[C@@]3(C)C[C@@H]23)c2c(C)cc(-c3cnc4cc(C)nn4c3)cc12]

A control used herein is an antibody manufactured using the published sequence of Crizanulumab, also known as ADAKVEO®, which is an anti-P-selectin antibody and an FDA approved product for the prevention of VOC in SCD. Crizanulumab has the CAS #1690318-25-2 and FDA Drug No. L7451S9126.

Antibody 8110 is a human anti-C5 recombinant antibody (clone 8110). This antibody is commercially available (e.g., Creative Biolabs #HPAB-1796LY).

### EXAMPLES

The following are examples of the methods of the disclosure. It is understood that various other embodiments may be practiced, given the general description provided above.

In the Examples section and elsewhere, representative types of antibodies which are useful in carrying out various embodiments of the disclosure are provided, e.g., with information on the particular vendor and/or catalog number. It should be understood that the disclosure is not limited to the exemplary embodiments which utilize antibody detection regents from a particular vendor/manufacturer. Antibodies against the biomarkers/analytes of the disclosure can be obtained from any manufacturer, including, Biolegend (San Diego, CA), Southern Biotech (Birmingham, AL), United States Biological (USB; Salem, MA), Lifespan Biosciences (LSBIO; Seattle, WA), Abcam (Cambridge, United Kingdom), Cell Signaling Technology (Danvers, MA), and Sigma-Aldrich (St. Louis, MO). Antibodies may also be generated using conventional techniques, e.g., immunization of a mammal such as a mouse or rabbit and/or hybridoma technology.

Examples 1-3 describe experiments and results that demonstrate the efficacy of complement inhibition using in vitro assays that model mechanisms of disease pathophysiology associated with SCD. In these experiments, a comparative analysis was performed that included the anti-properdin/anti-human serum albumin bispecific VHH antibody (ALXN1820), the small molecule factor D inhibitor (ALXN2050), and a monoclonal antibody inhibitor of C5 (N19/8). Additionally, these inhibitors were benchmarked to an antibody having the sequence of Crizanlizumab, a recently FDA approved therapeutic for the prevention of VOC in SCD.

The test articles for Examples 1-3 are shown in Table 1.

TABLE 1

| Molecule | Manufacturer | Lot # | Concentration |
|---|---|---|---|
| ALXN1820 | Alexion | 20190522PBS. | 12.1 mg/mL |
| | | Date of Manufacture: | |
| | | 2019 May 22 | |
| N19/8 | Alexion | PTN0090; | 2.65 mg/mL |
| | | 08 Jun. 2012 | |
| Crizanlizumab* | Invitrogen | 2899557 | 8.95 mg/mL |
| ALXN2050 | Alexion | 3; 22 Jun. 2016 | 20 mM |
| | (Achillion) | | |

*an antibody manufactured using the sequence of Crizanlizumab was used in the experiments described herein

### Example 1: Complement Induced Deposition Assay for C3 and C5b-9

### Induction of Complement Deposition on SS-RBCs by Heme and Assessment of ALXN1820 Blockade

RBCs and serum from SCD patients homozygous for the mutation in the hemoglobin gene (SS) were obtained from BioIVT (cat HUMANRBCALSUZN and HMRBC-SCA respectively) and from Sanguine Biosciences (Study #24348). Gelatin Veronal Buffer (GVB) was obtained from Boston Bioproducts (cat. IBB-300X). Mg-EGTA (cat. B106), C8 depleted normal human serum (cat. A325) and normal human serum (cat. NHS) were obtained from Complement Technology. PBS was obtained from Corning, cat. 21-031-CV. Porcine heme (Sigma, cat. 51280) was used at various concentrations (50-800 uM) to amplify complement activation and induce deposition on human cells.

All centrifugations were performed at 440×g for 5 min at 4° C. and supernatants aspirated with multi-channel pipets to avoid disturbing the loose RBC pellets.

### Identification of Appropriate Concentration of Heme for Use in Complement Inhibition Assays

Patient SS-RBCs were washed three times in PBS, resuspended in GVB, 5 mM Mg-EGTA and re-distributed to sterile V-bottom 96 well plates at a concentration of 2×10⁶ cells/well. Autologous serum was added to 20% final concentration. Heme was used at 0, 100, 200, 400, and 800 μM. Following incubation for 20-30 minutes at 37° C. 5% CO₂, PBS containing 10% EDTA (Corning, cat. 46-030-CI) was added to stop complement activation. RBCs were washed and stained with antibody to iC3b as detailed below.

### AP Blockade of Complement Deposition on SS-RBCs Induced by Heme

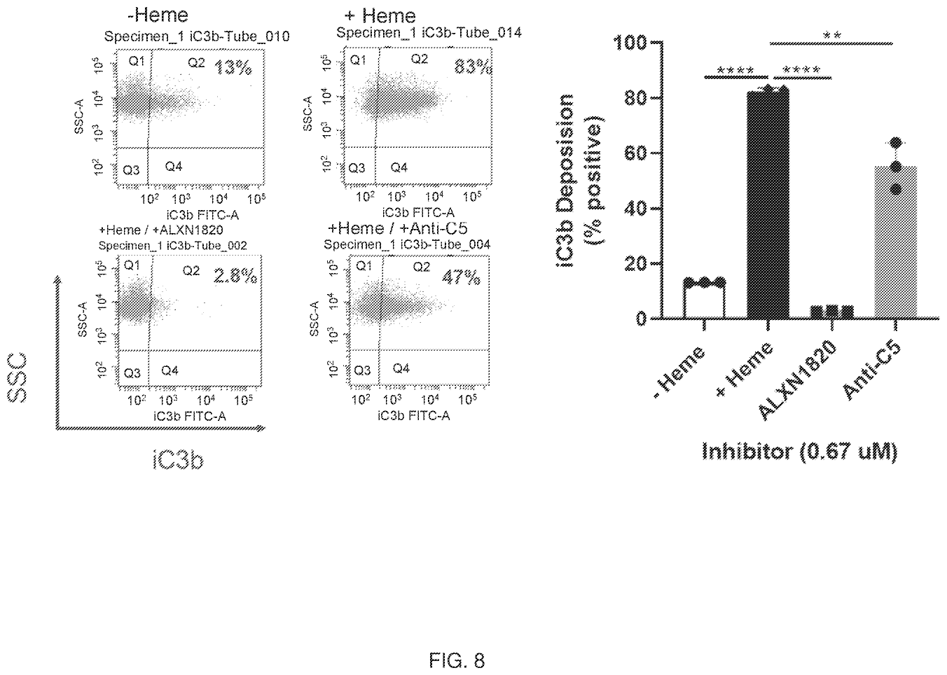

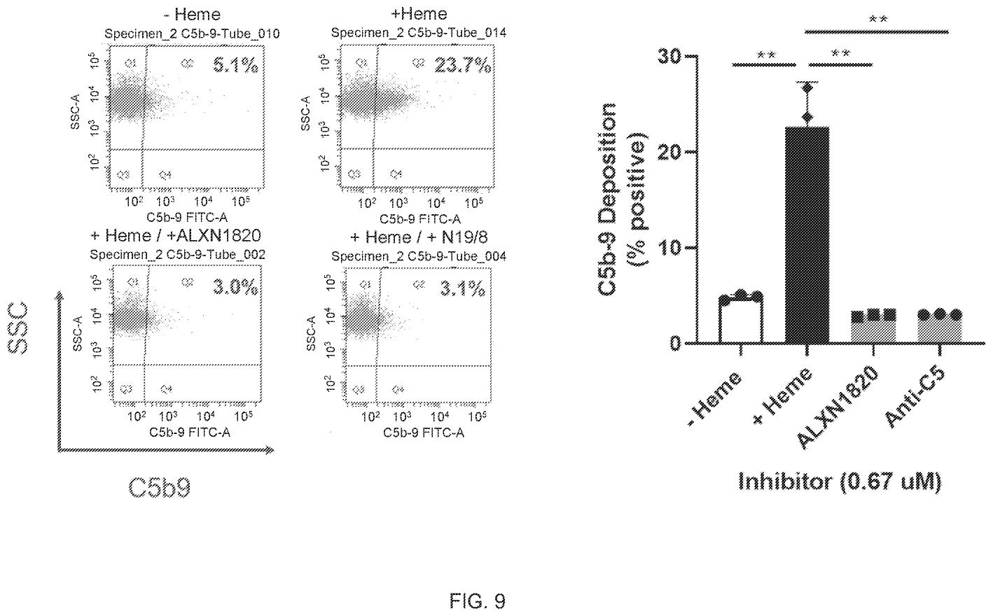

Patient SS-RBCs were washed two or three times in PBS. To induce complement deposition, RBCs were resuspended in GVB, 5 mM Mg-EGTA (assay buffer) at 5×10⁷ cells/mL and 30 μL added to sterile V-96 wells. Autologous serum was added to 20% final concentration. Alternatively, normal human serum was added to 20% final concentration (for data shown in FIG. 8 and FIG. 9). Complement inhibitors were diluted in assay buffer at 5× working stock of 3.125 μM and 10 μL added to wells containing cells. Porcine heme was added to 400 μM and the cells incubated for 20-30 minutes at 37° C., 5% CO₂. Complement activation was stopped by the addition of 150 μL/well PBS containing 10 mM EDTA. Cells were centrifuged and washed once with 200 μL PBS and stained for iC3b and C5b-9 deposition below.

Flow cytometric Analysis of iC3b and C5b-9 Deposition on the Surface of SS-RBCs

Cells were resuspended in 50 μL per well iC3b (Quidel, cat. A209) or C5b-9 antibody (Quidel, cat. A239) diluted to 4 μg/mL in PBS and incubated for 20-30 min at 4° C. (in some cases, staining for flow cytometry was performed in sheath fluid). Cells were washed twice with 150-200 μL PBS, resuspended in 50 μL goat anti-mouse IgG (H+L)-AF488 (Invitrogen cat. A11029) diluted to 4 μg/mL in PBS and incubated for 20-30 min at 4° C. In some experiments, goat anti-mouse IgG2b AF488 was used at 4 μg/mL (Invitrogen, cat. A21141). Cells were washed twice with 150-200 μL PBS and acquired on the LSR Fortessa for flow cytometric analysis.

### Results

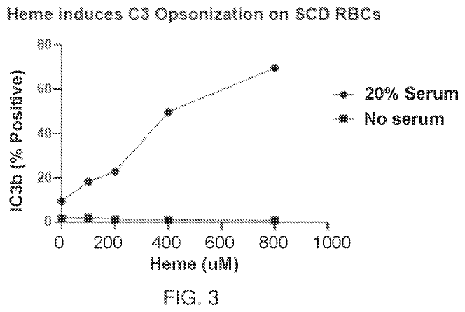

To determine if heme plays a role in the induction of C3 deposition and to find an optimal concentration for use in inhibition assays, SS-RBCs were incubated with various concentrations of heme (100-800 μM) in the presence of 20% autologous serum diluted in GVB containing MgEGTA. As shown in FIG. 3 and Table 2, flow cytometric analysis revealed that heme induced a potent and dose dependent opsonization of the SS-RBCs. As shown in FIG. 3, no staining was observed in the absence of serum indicating that antibody binding to the cells was not a result of cell damage, a common source of non-specific antibody staining in flow cytometric experiments. Similar experiments performed in GVB/10 mM EDTA resulted in no opsonization of SS-RBCs confirming that the staining was due to complement activation and not an artifact of assay conditions. The concentration of heme chosen to proceed with in the RBc and endothelial cell assays was 200 uM.

TABLE 2

| | | Q2 % Parent iC3b+ |
|---|---|---|
| Condition | Sample # | 20% SS-Serum |
| 0 Heme | Tube_031 | 9.6 |
| 100 uM heme | Tube_032 | 18.3 |
| 200 uM Heme | Tube_033 | 22.8 |
| 400 uM heme | Tube_034 | 49.6 |
| 800 uM heme | Tube_035 | 69.7 |
| | | Q2 % Parent iC3b+ |
| Condition | Sample # | No Serum |
| 0 Heme | Tube_036 | 1.7 |
| 100 uM heme | Tube_037 | 2 |
| 200 uM Heme | Tube_038 | 1.3 |
| 400 uM heme | Tube_039 | 1.1 |
| 800 uM heme | Tube_040 | 0.8 |

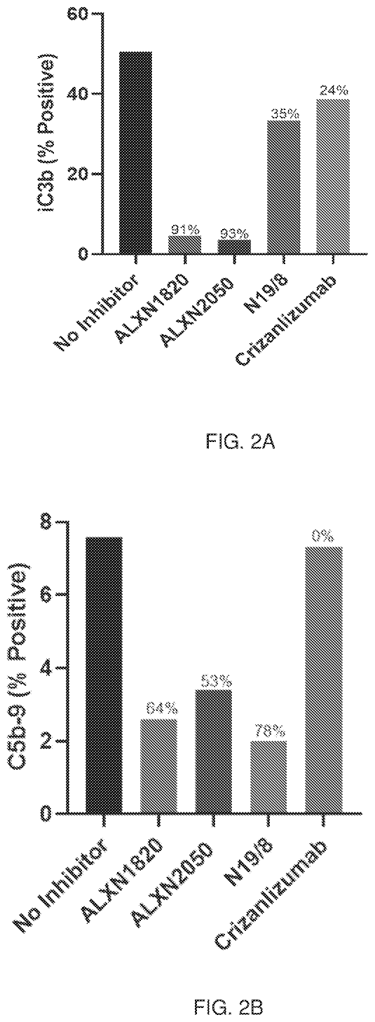

FIG. 2A and FIG. 2B show benchmarking of alternative pathway inhibitors, including ALXN1820, ALXN2050, and N19/8, against an antibody having the sequence of Crizanlizumab in heme-induced complement deposition on red blood cells. FIG. 2A shows heme-induced complement deposition of red blood cells for the protein C3. FIG. 2B shows heme-induced complement deposition of red blood cells for the protein C5b-9. FIG. 2A and FIG. 2B show that the antibody having the sequence of Crizanlizumab has little to no effect on C3 opsonization or C5b-9 deposition.

As shown in FIG. 2A, FIG. 2B and Table 3, flow cytometric analysis of SS-RBCs incubated with heme and autologous serum demonstrate marked deposition of C3 and marginal deposition of C5b-9 Addition of the AP inhibitors ALXN1820 and ALXN1850 (Properdin and factor D inhibitors respectively) reduced C3 deposition by more than 90%. As expected, the C5 inhibitor N19/8 had no effect on C3 deposition as C5 is downstream of C3 in the complement cascade. Of note, as shown in in FIG. 2A and FIG. 2B, the anti-P-selectin antibody recently approved by the FDA to treat SCD patients had no effect on C3 deposition. While C5b-9 deposition on SS-RBCs was not as robust as C3 deposition, AP blockade reduced MAC by more than 50%. As expected, as shown in FIG. 2A and FIG. 2B, the C5 inhibitor, N19/8 blocked MAC deposition by more than 75% while the anti-P selectin antibody had no effect

TABLE 3

| | | Q2 % | | | Q2 % |
|---|---|---|---|---|---|
| | | Parent | | | Parent |
| Sample # | Inhibitor | iC3b+ | Sample # | Inhibitor | C5b-9+ |
| Tube_026 | No Inhibitor | 50.5 | Tube_082 | No Inhibitor | 7.6 |
| Tube_034 | ALXN1820 | 4.5 | Tube_114 | ALXN1820 | 2.6 |
| Tube_046 | ALXN2050 | 3.5 | Tube_126 | ALXN2050 | 3.4 |
| Tube_070 | N19/8 | 33.3 | Tube_150 | N19/8 | 2 |
| Tube_078 | Crizanlizumab | 38.6 | Tube_158 | Crizanlizumab | 7.3 |

The experiment demonstrated that proximal complement blockade inhibited both C3 and Membrane attack complex (MAC) deposition while the C5 inhibitor, N19/8, solely blocked MAC deposition. An antibody having the sequence of Crizanlizumab, the anti-P selectin antibody recently approved for the reducing the incidence of VOC in SCD patients, had no effect on deposition.

In further experiments carried out essentially as described above, as is shown in FIG. 8 and FIG. 9, heme triggered significant levels of iC3b and C5b-9 deposition on red blood cells from sickle cell patients. Significance levels of P<0.0001 and <0.01 for iC3b and C5b-9 respectively as determined by students t test were noted. Heme-triggered complement deposition on SCD RBCs was blocked in the presence of anti-properdin by >95% for iC3b and by >85% for C5b-9 (P<0.0001 and <0.01 respectively). C5 inhibition also resulted in reduced iC3b and C5b-9 deposition on SCD RBCs (P<0.01 for both).

### Example 2: AP Inhibitors Block Heme Induced Complement Deposition on HMEC-1 Cells

The endothelial cell line HMEC-1 was purchased from ATCC (CRL 3243) and expanded and banked at AcCellerate (Cat. CBA02, lot 92-190318FG01). This is a dermal microvascular endothelial cell line. Cells were used in experiments at passage <5.

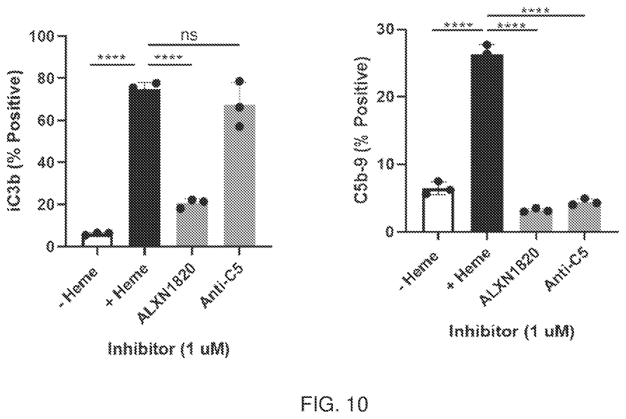

All centrifugation steps were performed for 5-7 min at 200-300 g at room temperature (RT). HMEC-1 cells were seeded into 6 well plates at 1.5×10⁵ cells per well in medium (Endothelial cell growth medium MV2, Promocell, cat. 22022) and allowed to reach confluency (72 hrs). Normal human serum (Complement Technologies, cat. NHS) was spiked with 1 uM inhibitor, diluted to 20% using Live cell imaging solution (LCIS) (Invitrogen, cat A1429DJ) containing 5-10 mM MgEGTA and added to HMEC-1 cultures in place of the medium. Alternatively, LCIS without MgEGTA was used as the test buffer (for data shown in FIG. 10). Heme was added to 400 μM, mixed and incubated for 20-30 min at 37° C. Cells were rinsed twice with 2 mLs PBS (Corning, cat. 21-031-CV) and detached with PBS containing 10 mM EDTA (Corning, cat. 46-034-CI). Cells were centrifuged, pellets resuspended in 400 μL sheath fluid (BD Biosciences, cat. 342003) and transferred to V-bottom 96 well plates in duplicate. After centrifugation, pellets were resuspended in 50 μL per well sheath fluid containing either iC3b or C5b-9 antibody diluted to 4 μg/mL. Following several washes, the cells were incubated with 50 μL of goat anti mouse IgG (H+L) AF 488 diluted to 4 μg/mL in sheath fluid for 30 min at 4° C. Following several washes, cells were acquired on the LSR Fortessa for flow cytometry analysis.

### Results

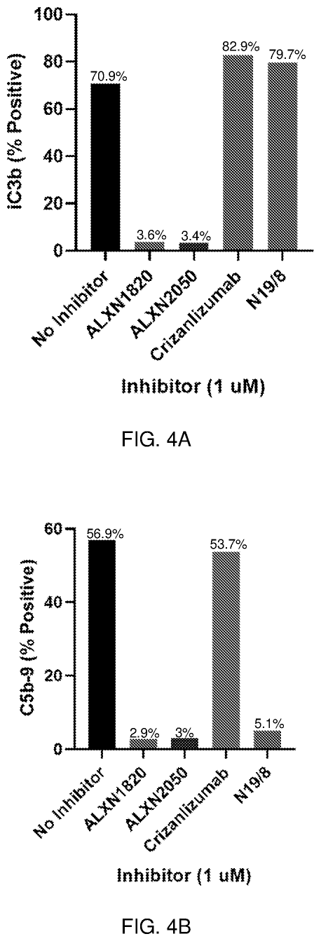

FIG. 4A and FIG. 4B show benchmarking of alternative pathway inhibitors, including ALXN1820, ALXN2050, and N19/8, against an antibody having the sequence of Crizanlizumab in heme-induced complement deposition on endothelial cells. FIG. 4A shows heme-induced complement deposition of endothelial cells for the protein C3. FIG. 4B shows heme-induced complement deposition of endothelial cells for the protein C5b-9. FIG. 4A and FIG. 4B show that ALXN1820 and ALXN2050 block heme-induced C3 and C5b-9 deposition on endothelial cells.

As seen in FIG. 4A, FIG. 4B, and Table 4, flow cytometric analysis of HMEC-1 cells incubated with heme and NHS demonstrate marked deposition of both C3 and C5b-9. Addition of the AP inhibitors ALXN1820 and ALXN1850 reduced C3 deposition by more than 90%. As expected, the C5 inhibitor N19/8 had no effect on C3 deposition. Similar to the experimental results obtained with SS-RBCs, the anti-P-selectin antibody had no effect on C3 deposition on endothelial cells, as shown in FIG. 4A and FIG. 4B. Unexpectedly, C5b-9 deposition was much more robust on HMEC-1 cells than on SS-RBCs. This may be due to the cleavage of EC surface regulators of complement upon mobilization of Weibal Palade bodies as reported (Frimat et al. (Frimat, Marie, Fanny Tabarin, Jordan D. Dimitrov, et al. 2013, Complement Activation by Heme as a Secondary Hit for Atypical Hemolytic Uremic Syndrome. Blood 122(2): 282-292)) leaving them more susceptible to deposition. Blockade of both proximal (C3) and terminal (C5) complement activation resulted in greater than 90% inhibition of MAC deposition. Notably, as shown in FIG. 4A and FIG. 4B, anti-P-selectin had no effect on TCC formation suggesting that crizanlizumab will not protect the SCD endothelium from complement mediated activation or damage.

TABLE 4

| | | Q2 % | | | Q2 % |
|---|---|---|---|---|---|
| | | Parent | | | Parent |
| Sample # | Inhibitor | iC3b+ | Inhibitor | Sample # | C5b-9+ |
| Tube_007 | No Inhibitor | 70.9 | No Inhibitor | Tube_017 | 56.9 |
| Tube_001 | ALXN1820 | 3.6 | ALXN1820 | Tube_011 | 2.9 |
| Tube_002 | ALXN2050 | 3.4 | ALXN2050 | Tube_012 | 3 |
| Tube_004 | Crizanlizumab | 82.9 | Crizanlizumab | Tube_014 | 53.7 |
| Tube_005 | N19/8 | 79.7 | N19/8 | Tube_015 | 5.1 |

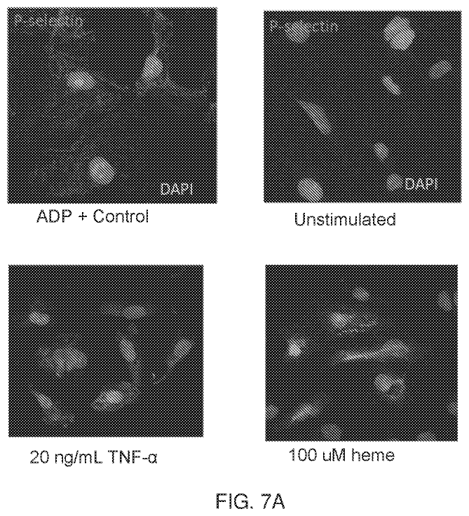

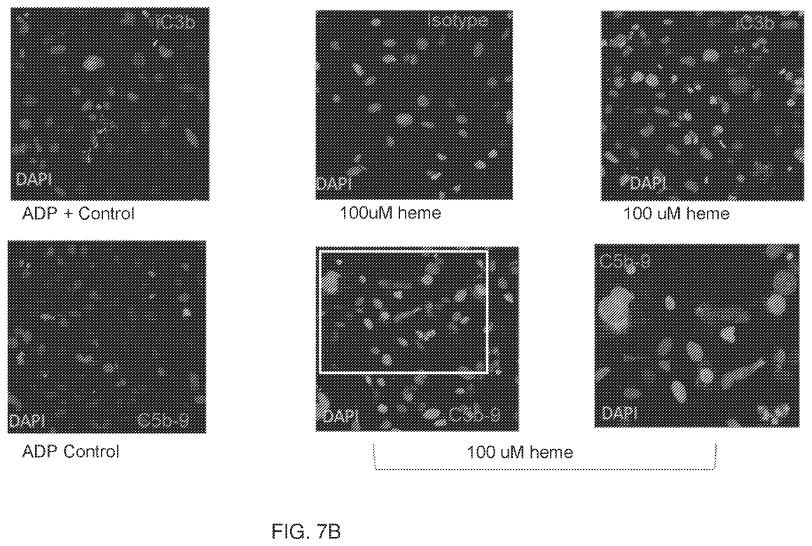

FIG. 7A and FIG. 7B show microscopic imaging of p-selectin upregulation and complement deposition on endothelial cells treated with heme, respectively. FIG. 7A shows that p-selectin is upregulated by sickle cell disease relevant agonists. FIG. 7B shows complement deposition is induced by heme.

The experiment demonstrated that proximal complement blockade inhibited both C3 and Membrane attack complex (MAC) deposition while the C5 inhibitor, N19/8, solely blocked MAC deposition. An antibody having the sequence of Crizanlizumab, the anti-P selectin antibody recently approved for the reducing the incidence of VOC in SCD patients, had no effect on deposition.

FIG. 10 shows the results of further experiments carried our essentially as described above. FIG. 10 features bar charts showing flow cytometry-based analyses of heme-induced complement fragment deposition on endothelial cells exposed to herne and the effect of anti-properdin and anti-C5 antibodies on complement deposition. Shown are changes in complement fragment levels, from left to right, normal, heme, heme+anti-properdin, and heme+anti-C5 pretreatment. The left-hand panel shows C3/C3b/iC3b deposition and the right-hand panel shows C5b9 deposition. As shown in FIG. 10, heme potently triggered deposition of iC3b and C5b-9 on HMEC-1 cells (P<0.0001 for both).

FIG. 10 shows that in the presence of anti-properdin antibody, deposition on HMEC-1 was blocked by >70% for iC3b and >85% for C5b-9. While C5 inhibition effectively blocked MAC deposition (P<0.0001), deposition of iC3b was not affected.

### Example 3: AP Inhibitors Prevent Heme Induced Upregulation of Tissue Factor Expression on Human Monocytes in Whole Blood

Human whole blood was obtained internally from healthy donors according to Institutional Review Board protocol. Blood was drawn into custom hirudin blood draw tubes obtained from Haematologic Technologies (cat. SCAT-296-5/5, 500 ATU/mL). This anti-coagulant allows simultaneous activation of complement and coagulation (through but not beyond thrombin). Blood was aliquoted into facs tubes (0.2 mL per tube) and inhibitors added to 1 μM. Heme was added to 100 μM and the samples were incubated for 4 hrs at 37° C., 5% CO₂, mixing approximately every 30 minutes. To stain for flow cytometry, 10 μL of TruStain FcX (Biolegend, cat. 422302) was added for a 5-10 minute incubation at RT (to block IgG receptors). Mouse anti-human CD14 PerCP Cy5.5 (BD Biosciences, cat. 550787) (5 μL per sample) and mouse anti-human tissue factor-PE (BD Biosciences, cat. 550312) were added (20 μL per sample) and the samples were mixed and incubated for 30 minutes at RT. To lyse RBCs, 3 mLs of 1× lysis buffer (BD Biosciences Cat. 555899) was added to each tube followed by vortexing and incubation for 20 min at RT. The samples were centrifuges at 340 g for 5 minutes and washed twice with PBS. Cell pellets were resuspended in 0.5 mL PBS and acquired on the LSR Fortessa for flow cytometric analysis. Monocytes were gated via CD14 triggering and data was expressed as percentage of CD14 monocytes positive for tissue factor (TF).

### Results

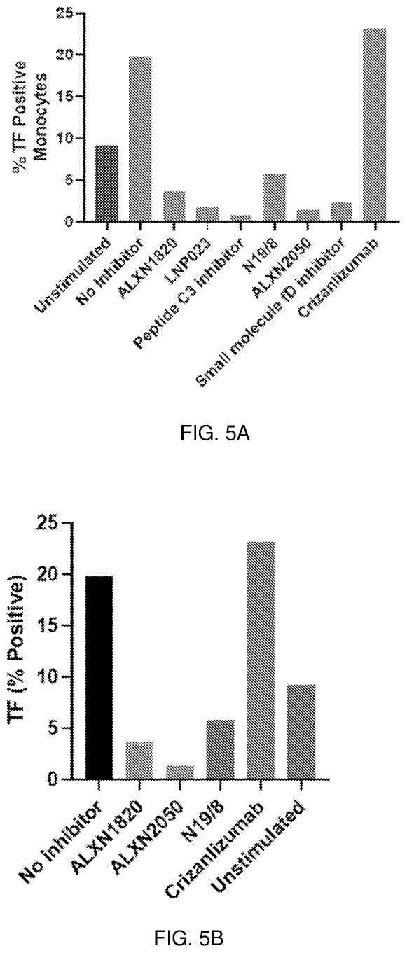

Heme is considered to be a potent DAMP (damage-associated molecular pattern) and a second hit for thrombotic disorders such as aHUS (atypical hemolytic uremic syndrome). In addition, multiple reports have indicated a role for C5a in thromboinflammation (Ekdahl et al. (Ekdahl, Kristina N., Teramura, Yuji, Hamad, Osama A., et al. 2016 Dangerous Liasons: Complement, Coagulation, and Kallikrein/Kinin Cross-talk as a Linchpin in the Events Leading to Thromboinflammation. Immunological Reviews 274:245-269), Thomas et al. (Thomas, Anub M., et al. 2019 Complement Component C5 and TLR Molecule CD14 Mediate Heme-Induced Thromboinflammation in Human Blood. J. Immunol 203: 1571-1578)). To address questions about the role for complement in heme induced thromboinflammation, a whole blood model was developed utilizing the anti-coagulant hirudin which allows both the activation of complement and thrombin. Human blood from healthy donors was incubated with heme and analyzed for monocyte tissue factor TF expression. TF is the initiator of the extrinsic coagulation cascade and is relevant in thrombosis and hemostasis. Complement activation by heme induces TF upregulation in monocytes. Downstream coagulation proteases amplify platelet and inflammation via protease-activated receptors (PARs). FIG. 5A and FIG. 5B shows benchmarking of complement inhibitors, including ALXN1820, LNPO23 (Iptacopan), a C3 peptide inhibitor, N19/8, ALXN2050, and a small molecule factor D (fD) inhibitor, against an antibody having the sequence of Crizanlizumab for the upregulation of TF. As seen in FIG. 5A, FIG. 5B and Table 5, flow cytometric analysis of monocytes from whole blood incubated with heme upregulated expression of TF. Samples incubated with inhibitors of both proximal and terminal complement pathway of complement blocked TF upregulation. Samples incubated with an antibody having the sequence of crizanlizumab had no effect on TF expression, as seen in FIG. 5A and FIG. 5B, suggesting that complement therapy would better address thrombosis in SCD. All complement inhibitors tested prevented the upregulation of TF induced by heme other than an antibody having the sequence of Crizanlizumab.

TABLE 5

| Inhibitor | Sample # | Q2 % Parent TF+ |
|---|---|---|
| No Inhibitor | Tube_008 | 19.8 |
| ALXN1820 | Tube_004 | 3.7 |
| ALXN2050 | Tube_005 | 1.4 |
| N19/8 | Tube_003 | 5.8 |
| Crizanlizumab | Tube_007 | 23.2 |

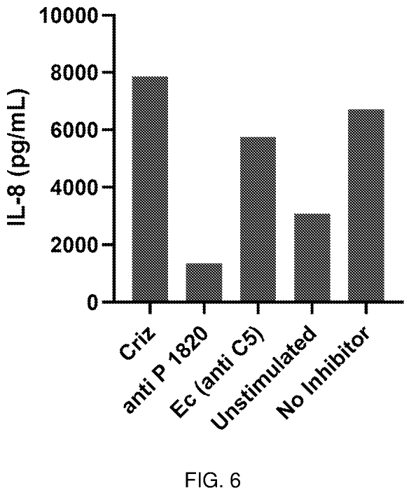

The chemokine IL-8 is a mediator of neutrophil migration and activation. IL-8 levels are elevated in patients with sickle cell disease and is associated with VOC. Heme triggered the induction of TF expression, the initiator of the extrinsic coagulation cascade, on monocytes and the elaboration of the inflammatory cytokine IL-8. FIG. 6 shows IL-8 levels in a whole blood model of thromboinflammation when exposed to alterative pathway inhibitors, including ALXN1820, and Ec (anti C5), and compared to an antibody having the sequence of Crizanlizumab. FIG. 6 shows that an antibody having the sequence of Crizanlizumab has no effect on IL-8 production in the whole blood model of thromboinflammation. ALXN1820 inhibited the production of IL-8 in the whole blood model of thromboinflammation, while an antibody having the sequence of Crizanlizumab had no effect.

### Example 4. Efficacy of Inhibition of Complement Activation in Heme-Triggered Vaso-Occlusion Crisis

This study used male Townes S/S mice on a 129/B6 mixed genetic background (Wu et al. 2006). In Townes S/S mice, mouse α- and β-globin gene loci are deleted and replaced by human α and ^{A}γβ^{S} globins. When carrying two copies of the β^{S} allele (ha/ha::β^{S}/β^{S}), mice develop a human sickle disease phenotype with sickle-shaped red blood cells (RBCs) seen in blood smears. Breeding pairs were obtained from the Jackson Laboratories. The animals were housed under conventional conditions at the Animal Care Facility at Imagine Institute.

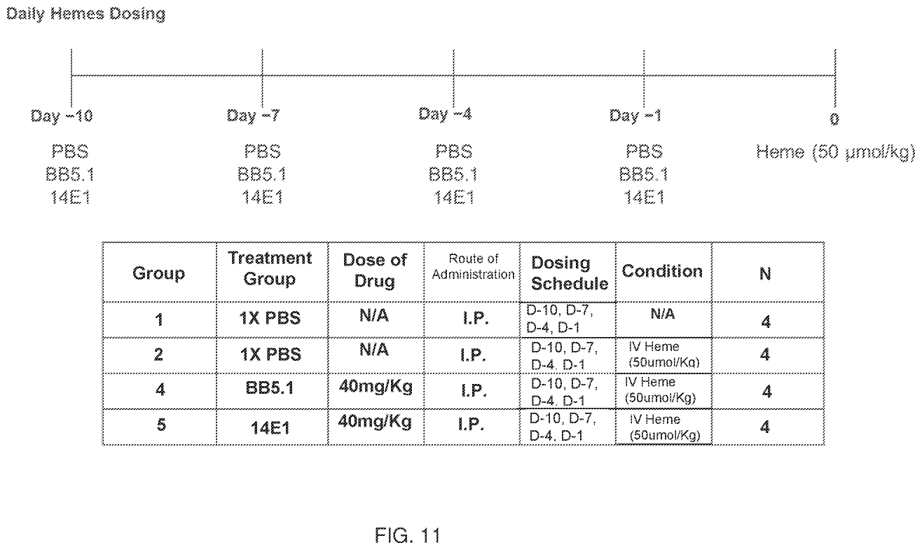

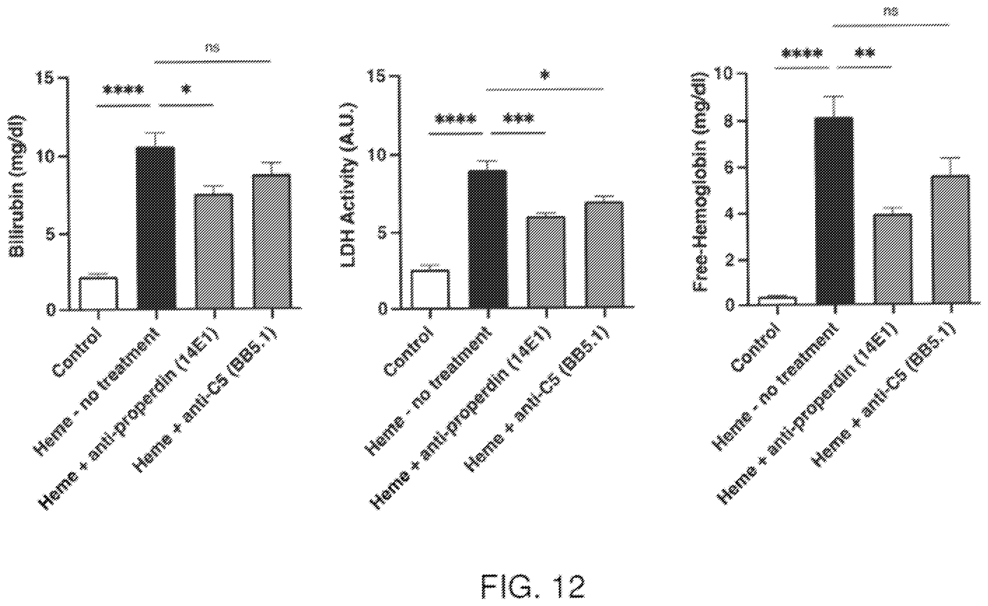

To demonstrate the efficacy of inhibition of complement activation in VOC, Townes SS mice are divided into five groups and prophylactically treated with PBS (vehicle), anti-properdin mAb, or anti-C5 mAb) four times from ten days before heme treatment (FIG. 11). The animals were exposed to 50 μmol/Kg heme for three hours after which the animals were sacrificed (FIG. 12). In one of the vehicle-treated group, animals were not exposed to heme and serve as a baseline (FIG. 12). Upon euthanasia, blood samples and critical organs were harvested from animals to measure the level of complement deposition on RBCs, intravascular hemolysis and the severity of vaso-occlusions (FIG. 12). Mice were phlebotomized by retro-orbital bleeding using a capillary tube internally coated with heparin/EDTA anticoagulant. Mice were euthanized by cervical dislocation and perfused with 1 mL of saline solution through the left ventricle. Lung, liver, kidney and spleen were collected and weighed.

Plasma heme was measured using Hemin Assay Kit (Sigma-Aldrich reference MAK036), determined by a coupled enzyme reaction, which results in a colorimetric (570 nm) product, proportional to the hemin present in plasma. Plasma was diluted 1:4 with hemin assay buffer to a final volume of 50 μL. The reaction mix was prepared in duplicate in the following order: 3 μL enzyme mix, 2 μL hemin substrate, 43 μL hemin assay buffer and 2 μL hemin probe. Hemoproteins present in the plasma can generate a background signal, so to control for this variable, a blank was prepared for each sample by omitting the enzyme from reaction mix. The reaction mix was added to samples in a 96 well-plate, homogenized using a horizontal shaker and incubated 30 minutes at room temperature, protected from light. A hemin standard solution was prepared in the 96-well plate by diluting the hemin standard provided in the kit. Absorbance was measured at 570 nm in kinetic mode using an Infinite F200 Pro multimode plate reader (Tecan). The background signal was removed by subtracting the blank sample value from each sample reading to obtain the corrected measurement. The hemin concentration was determined by plotting the corrected measurement to a standard curve.

The level of intravascular hemolysis was determined by multiple measures including total bilirubin, plasma lactate dehydrogenase (LDH) activity, and free hemoglobin. Exposure of the SCD animals to heme triggered intravascular hemolysis, which is effectively prevented by pretreatment with anti-properdin or anti-C5 antibodies (FIG. 12).

Plasma bilirubin was measured using a Bilirubin Assay Kit (Sigma-Aldrich reference MAK126), based on the Jendrassik-Grof method. This method was based on the reaction of bilirubin with diazotized sulfanilic acid, resulting in a colorimetric product measured at 530 nm, proportionate to the bilirubin present in the sample. Total bilirubin was determined by the addition of Reagent C containing caffeine benzoate which splits bilirubin from the unconjugated bilirubin-protein complex. Plasma was diluted 1:2 with PBS to a final volume of 50 μL. Work reagent was prepared in the following order: 50 μL reagent A, 20 μL reagent B and 130 μL reagent C. A blank was prepared for each sample by omitting the reagents B and C from the reaction mix (replaced by saline solution). The reaction mix was added to samples in a 96 well-plate, homogenized using a horizontal shaker and incubated 10 minutes at room temperature, protected from light. Absorbance was measured at 530 nm using an Infinite F200 Pro multimode plate reader (Tecan). Background was removed by subtracting the blank sample value from each sample reading to obtain the corrected measurement. Bilirubin concentration was determined by the following equation: [(Sample−Blank)/(Calibrator−Water)]×5 mg/dL.

Whole blood was collected on K2 EDTA tubes (Melet Schloesing Laboratoires). Cells were removed from plasma by centrifugation for 15 minutes at 2,000×g using a refrigerated centrifuge. This step also depletes platelets in the plasma sample. Plasma was apportioned into 50 μL aliquots and stored at −80° C.

Plasma LDH was measured using a Pierce LDH Cytotoxicity Assay Kit (Thermofisher Scientific reference 88953). Reaction mix was prepared by combining 0.6 mL of assay buffer with 11.4 mL of substrate mix in a 15 mL conical tube. Plasma was diluted 1:2 with PBS to a final volume of 50 μL. Reaction mix was added to samples in a 96 well-plate, homogenized using a horizontal shaker and incubated 30 minutes at room temperature, protected from light. The reaction was stopped by adding 50 μL of stop solution to each sample. Absorbance was measured at 490 nm and 680 nm using an Infinite F200 Pro multimode plate reader (Tecan). LDH activity was determined as [(LDH 490 nm)−(LDH 680 nm)].

Plasma hemoglobin was measured using Drabkin's Reagent (Sigma-Aldrich reference D5941). This procedure was based on the oxidation of hemoglobin and its derivatives (except sulfhemoglobin) to methemoglobin in the presence of alkaline potassium ferricyanide. Methemoglobin reacts with potassium cyanide to form cyanmethemoglobin, which had maximum absorption at 540 nm. The color intensity measured at 540 nm is proportional to the total hemoglobin concentration. Plasma was transferred to a 96 well-plate (20 μL for each sample). Drabkin's solution was prepared by reconstituting one vial of the Drabkin's reagent with 1,000 mL of water and 0.5 mL of 30% Brij L23 Solution, (Sigma Catalog Number B4184). Drabkin's solution (180 μL) was added to samples in a 96 well-plate, homogenized using a horizontal shaker and incubated 15 minutes at room temperature, protected from light. Hemoglobin calibration curve was prepared in Drabkin's solution. Absorbance was measured at 540 nm using an Infinite F200 Pro multimode plate reader (Tecan). Background was removed by subtracting the blank sample value from each sample reading to obtain the corrected measurement. Hemoglobin concentration was determined by plotting the corrected measurement to a calibration curve.

Blood (45 μL) was incubated with 5 μL of mouse FcR Blocking Reagent (Miltenyi Biotec reference 130-092-575) for 10 minutes and diluted 1:2 with 50 μL of cell staining buffer (Biolegend reference 420201). Samples were then stained with antibodies against Ter-119 Pacific Blue (Biolegend reference 116232; 1/100 dilution), mouse TfR1/CD71 PerCP/Cy5.5 (Biolegend reference 113816; 1/100 dilution), C5b9-FITC (Santa Cruz Biotechnologies reference sc-66190 FITC; 1/20 dilution) or C3-FITC (Cedarlane reference CL7631F; 1/50 dilution). Dead cells were excluded by Live-Dead (eBioscience).

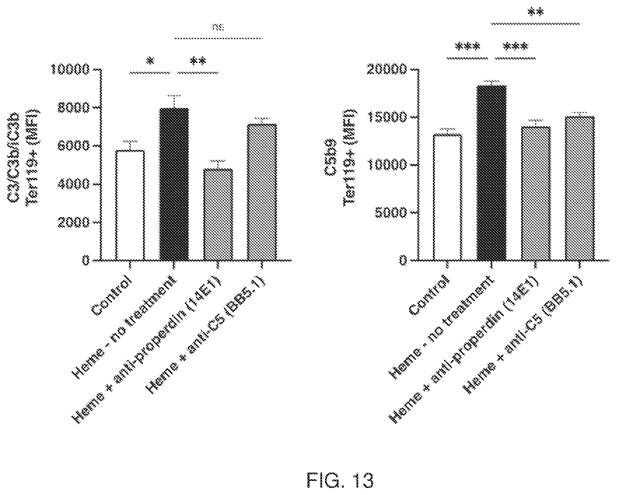

Cells were further analyzed by flow cytometry (Gallios Beckman Coulter) using FlowJo software (Tree Star). Flow cytometry-based SS RBC analyses revealed marked increase in both C5b9 and C3 deposition on SS RBCs upon exposure to heme (FIG. 13). Pretreatment with either BB5.1 (anti-C5) or 14E1 (anti-properdin) nearly completely prevented the increase in C5b9 deposition on SS RBCs (FIG. 13). As C5b9 staining represents potential membrane attack complex (MAC) formation, prevention of C5b9 deposition is expected to reduce complement-mediated intravascular hemolysis (FIG. 13). Next, C3 deposition was then measured on SS RBCs. The increase in C3 deposition upon exposure to heme was markedly reduced by 14E1 (anti-properdin) while anti-C5 did not reduce the level of C3 opsonization (FIG. 13).

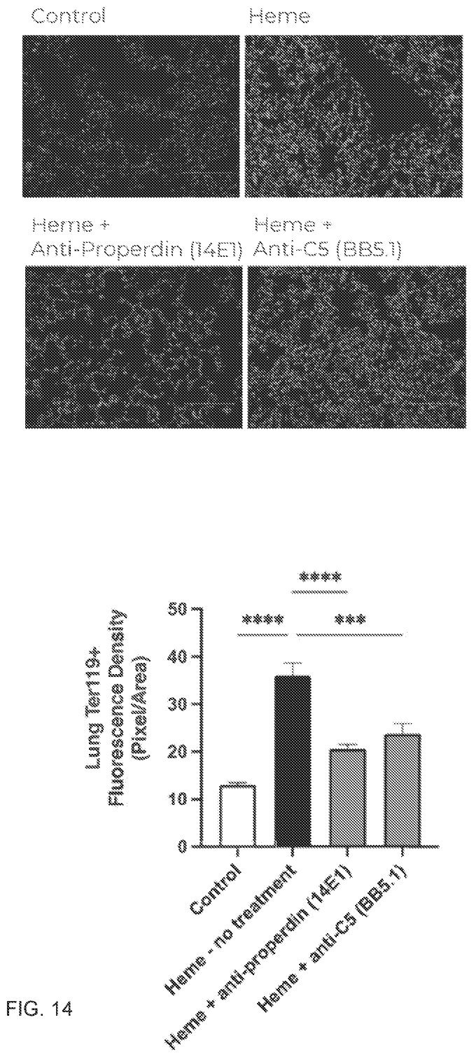

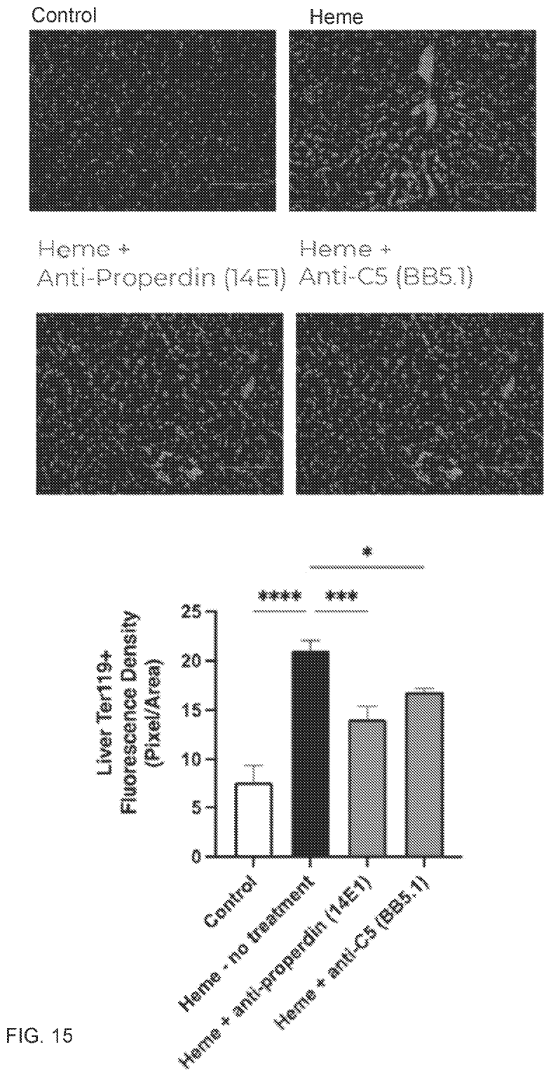

Paraffin-embedded lung, spleen, liver or kidney sections (5 μm) were processed for deparaffinization, rehydration and antigen retrieval using a citrate buffer for 20 minutes at 95° C. (Biolegend reference 928502). Samples were delimited with a PAP-pen, blocked 15 minutes with high protein IHC/ICC blocking buffer (eBioscience reference 00-4952-54) and then incubated 1 hour with primary antibodies against Ter-119, a marker for vessel-trapped RBCs, coupled to alexa fluor-488 (Biolegend reference 116215; 1/100 dilution). Slides were washed thoroughly with TBS Tween-20 0.05% for 3×10 minutes and mounted with prolong diamond antifade mountant with DAPI (ThermoFischer Scientifc reference P36962). Images were acquired on an EVOS M5000 Imaging System (ThermoFisher Scientific) at magnification×200 and positive pixels per area were analyzed using ImageJ software. The intensity of vaso-occlusion was visualized and quantified by immunofluorecence (IF) staining of RBCs (Ter-119) clogging the vessels in vital organs including the lung and liver (FIG. 14 and FIG. 15). Exposure of the SCD mice to heme markedly increased the intensity of vaso-occlusion in the lung and liver. Pretreatment with either anti-properdin (14E1) or anti-C5 (BB5.1) effectively reduced the level of vaso-occlusion in a statistically significant manner in comparison to PBS treatment. No clear differences were observed between BB5.1 and 14E1 treatment group.

Statistical analyses studies were performed using a one-way analysis of variance (ANOVA) test followed by a Tukey's test (multiple comparison test) or Kruskal-Wallis test (non-parametric) for analysis of treatment effect versus controls. All statistical analyses were derived using GraphPad software (v 6.00, San Diego, California, USA). Statistical significance to reject the null hypothesis was identified at the P<0.05 level. For illustrative purposes, significance levels of P<0.01 and P<0.005 were also noted.

### OTHER EMBODIMENTS

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries (e.g., PUBMED, NCBI, FDA Drug, or UNIPROT accession numbers), and other references mentioned herein are incorporated by reference in their entirety. In case of conflict, the present specification, including definitions, will control.

While particular embodiments of the present disclosure have been illustrated and described, it would be obvious to those skilled in the art that various other changes and modifications may be made without departing from the spirit and scope of the disclosure. It is therefore intended to cover in the appended claims all such changes and modifications that are within the scope of this disclosure.

What is claimed is:

1. A method of identifying a test compound for treating sickle cell disease (SCD), β-thalassemia (BT), or sickle cell BT, the method comprising,
contacting a test sample comprising cells with heme, serum, and the test compound; and
measuring a biological phenomena comprising
(1) deposition of a complement factor on the cells in the test sample; or
(2) effect(s) of the complement factor deposition of on target effector cells;
wherein an attenuation in the biological phenomena in the test sample compared to the biological phenomena in a reference standard is indicative that the test compound is effective in treating sickle cell disease (SCD), β-thalassemia (BT), or sickle cell BT.

2. The method of claim 1, wherein the test sample comprises red blood cells (RBCs), endothelial cells, or blood cells, or a combination thereof.

3. The method of claim 2, wherein the blood cells are monocytes, neutrophils, and/or platelets.

4. The method of claim 1, wherein deposition of the complement factor on RBCs and/or endothelial cells is measured.

5. The method of claim 1, wherein the target effector cells are endothelial cells or blood cells.

6. The method of claim 5, wherein the blood cells are monocytes, neutrophils and/or platelets.

7. The method of claim 1, wherein the reference standard comprises an experimentally measured or predetermined level of a signal for the biological phenomena in a control sample that is devoid of the test compound.

8. The method of claim 7, wherein the signal for the biological phenomena is a baseline C3 positivity level and/or a baseline C5b9 positivity level of about or greater than 20% in a population of endothelial cells.

9. The method of claim 8, wherein the baseline C3 positivity level and/or a baseline C5b9 positivity level in the population of endothelial cells is greater than 30%.

10. The method of claim 8, wherein the baseline C3 positivity level and/or a baseline C5b9 positivity level in the population of endothelial cells is greater than 50%.

11. The method of claim 7, wherein the signal for the biological phenomena is a baseline Tissue Factor (TF) positivity level of about or greater than 10% in monocytes.

12. The method of claim 11, wherein the baseline TF positivity level in monocytes is greater than 15%.

13. The method of claim 11, wherein the baseline TF positivity level in monocytes is greater than 20%.

14. A method of identifying a test compound for treating sickle cell disease (SCD), β-thalassemia (BT), or sickle cell BT, the method comprising,
(a) contacting a first sample comprising cells with heme and serum;
(b) contacting a second sample comprising the cells with the test compound, heme and serum, and
(c) measuring a biological phenomena comprising (1) deposition of a complement factor on the cells in said first and the second samples; or (2) effect(s) of the complement deposition in the cells of said first and second samples on target effector cells;
wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

15. The method of claim 14, wherein the first sample comprises red blood cells (RBCs) or endothelial cells, or a combination thereof.

16. The method of claim 15, wherein the blood cells are monocytes, neutrophils, and/or platelets.

17. The method of claim 14, wherein the second sample comprises RBCs or endothelial cells, or a combination thereof.

18. The method of claim 17, wherein the blood cells are monocytes, neutrophils, and/or platelets.

19. The method of claim 14, wherein the target effector cells are endothelial cells or blood cells, or a combination thereof.

20. The method of claim 19, wherein the blood cells are monocytes, neutrophils and/or platelets.

21. The method of claim 14, wherein the biological phenomena is a baseline C3 positivity level and/or a baseline C5b9 positivity level of about or greater than 20% in a population of endothelial cells.

22. The method of claim 21, wherein the baseline C3 positivity level and/or a baseline C5b9 positivity level in the population of endothelial cells is greater than 30%.

23. The method of claim 21, wherein the baseline C3 positivity level and/or a baseline C5b9 positivity level in the population of endothelial cells is greater than 50%.

24. The method of claim 14, wherein the biological phenomena is a baseline Tissue Factor (TF) positivity level of about or greater than 10% in monocytes.

25. The method of claim 24, wherein the baseline TF positivity level in monocytes is greater than 15%.

26. The method of claim 24, wherein the baseline TF positivity level in monocytes is greater than 20%.

27. A method of identifying a test compound for treating sickle cell disease (SCD), β-thalassemia (BT), or sickle cell BT, the method comprising,
(a) contacting a first sample comprising RBCs with heme and serum;
(b) contacting a second sample comprising the RBCs with the test compound, heme, and serum, and
(c) measuring a biological phenomena comprising (1) deposition of a complement factor on the RBCs in said first and the second samples;
wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

28. A method of identifying a test compound for treating sickle cell disease (SCD), β-thalassemia (BT), or sickle cell BT, the method comprising,
(a) contacting a first sample comprising endothelial cells (ECs) with heme and serum for a period sufficient to induce complement deposition on ECs;
(b) contacting a second sample comprising the ECs with the test compound, heme, and serum, and
(c) measuring a biological phenomena comprising (1) deposition of a complement factor on the ECs in said first and the second samples; or (2) effect(s) of the complement deposition in the ECs of the first and second samples on target effector cells comprising ECs;
wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

29. A method of identifying a test compound for treating sickle cell disease (SCD), β-thalassemia (BT), or sickle cell BT, the method comprising,
(a) contacting a first sample comprising blood cells such as monocytes, neutrophils, and/or platelets with heme and serum;
(b) contacting a second sample comprising the blood cells with the test compound, heme, and serum, and
(c) measuring a biological phenomena comprising effect(s) of deposition of a complement factor on the blood cells in said first and the second samples on target effector cells comprising blood cells;
wherein an attenuation in the biological phenomena of (c) in the second sample compared to the biological phenomena of (c) in the first sample is indicative that the test compound is effective in treating sickle cell disease (SCD), β-thalassemia (BT), or sickle cell BT.

30. The method of any one of the preceding claims, wherein the measuring step comprises flow cytometry.

31. The method of any one of the preceding claims, wherein the complement factor comprises complement factor C3, or a fragment thereof, or complement factor C5b9.

32. The method of claim 31, wherein the complement factor is complement factor C3.

33. The method of claim 31, wherein the complement factor is a protein fragment of complement factor C3.

34. The method of claim 33 wherein the protein fragment of complement factor C3 is iC3b.

35. The method of any one of the preceding claims, wherein the heme comprises free heme provided at a concentration of 200 μM.

36. The method of any one of the preceding claims, wherein the RBCs comprise sickle cell RBCs (ssRBC) or RBCs induced to form a ssRBC phenotype.

37. The method of claim 36, wherein the RBCs induced to form a ssRBC phenotype comprise cell-surface expression of phosphatidylserine (PS) or phosphatidylethanolamine (PE).

38. The method of claim 36, wherein the RBCs comprise ssRBCs obtained from a sickle cell patient.

39. The method of any one of the preceding claims, wherein the serum comprises autologous serum.

40. The method of claim 39, wherein the serum is from a sickle cell patient.

41. The method of any one of claims 2, 4, 5, 15, 17, 19, or 28, wherein the endothelial cells comprise dermal microvascular endothelial cells.

42. The method of any one of the preceding claims, wherein the effect(s) of complement deposition on target effector cells is mediated via a complement receptor (CR) in effector cells.

43. The method of claim 42, wherein the complement receptor is CR3 in monocytes.

44. The method of any one of the preceding claims, wherein the effect(s) of complement deposition on target effector cells results in upregulation of tissue factor (TF).

45. The method of any one of claims 1-13, wherein the sample comprises a blood sample.

46. The method of claim 45, wherein the blood sample is a whole blood sample.

47. The method of claim 45 or 46, wherein the blood sample comprises an anti-coagulant.

48. The method of claim 47, wherein the anti-coagulant is hirudin.

49. The method of any one of claims 14-34, wherein the first and/or the second sample comprises a blood sample.

50. The method of claim 49, wherein the blood sample is a whole blood sample.

51. The method of claim 49 or 50, wherein the blood sample comprises an anti-coagulant.

52. The method of claim 51, wherein the anti-coagulant is hirudin.

53. The method of any one of the preceding claims, wherein the method further comprises contacting a third sample comprising cells that have been contacted with heme and blood with an inhibitor of the complement alternative pathway (CAP).

54. The method of claim 53 wherein the cells are red blood cells (RBCs), endothelial cells (ECs), or blood cells.

55. The method of claim 54, wherein the blood cells are monocytes, neutrophils and/or platelets.

56. The method of any one of claim 53-55, wherein the CAP inhibitor comprises a C3 inhibitor, a factor P inhibitor, a factor D (FD) inhibitor, or a C5 inhibitor.

57. The method of claim 56, wherein the CAP inhibitor comprises a peptide inhibitor of C3 or an oral factor D inhibitor.

58. The method of claim 56, wherein the CAP inhibitor comprises a properdin inhibitor.

59. The method of claim 58, wherein the properdin inhibitor is an anti-properdin antibody.

60. The method of claim 59, wherein the anti-properdin antibody is a bispecific antibody.

61. The method of claim 59 or 60, wherein the bispecific anti-properdin antibody is a mini-body.

62. The method of claim 56, wherein the CAP inhibitor comprises a C5 inhibitor.

63. The method of claim 62, wherein the C5 inhibitor is an anti-C5 antibody.

64. The method of claim 63, wherein the anti-C5 antibody is eculizumab, ravulizumab, antibody 8110, or antibody N19-8 that binds specifically to human C5.

65. The method of claim 64, wherein the anti-C5 antibody is a bispecific antibody.

66. The method of claim 63 or 65, wherein the bispecific anti-C5 antibody is a mini-body.

67. The method of any one of the preceding claims, wherein the method further comprises contacting a control sample comprising cells that have been contacted with heme and blood with an inhibitor of P-selectin.

68. The method of claim 67, wherein the cells are red blood cells (RBCs), endothelial cells (ECs), or blood cells, or a combination thereof.

69. The method of claim 68, wherein the blood cells are monocytes, neutrophils and/or platelets.

70. The method of any one of claims 67-69, wherein the inhibitor of P-selectin is an antibody that binds to P-selectin.

71. The method of claim 70, wherein the antibody is a monoclonal antibody.

72. The method of claim 70 or 71, wherein the antibody is crizanlizumab.

73. The method of any one of the preceding claims, wherein the contacting comprises administering heme, serum, and the test compound to a test animal.

74. The method of claim 73, wherein the test animal is a mouse, rat, guinea pig, rabbit, hamster, sheep, goat, monkey, or primate.
